(19) United States
(12) Patent Application Publication
(10) Pub. No.: US 2022/0168424 A1
(43) Pub. Date: Jun. 2, 2022

(54) THERMALLY STABLE ANTIBACTERIAL QUATERNARY AMMONIUM NANOPARTICLES

(71) Applicant: NOBIO LTD., Kadima (IL)

(72) Inventors: Nathan ZALTSMAN, Hadera (IL); Ervin Itzhak WEISS, Herzliya (IL)

(73) Assignee: NOBIO LTD., Kadima (IL)

(21) Appl. No.: 17/434,200

(22) PCT Filed: Feb. 27, 2020
(86) PCT No.: PCT/IL2020/050228

§ 371 Date: Aug. 26, 2021

Related U.S. Application Data
(60) Provisional application No. 62/811,121, filed on Feb. 27, 2019.

(51) Int. Cl. A61K 47/18; A61K 47/69; A61K 31/14; A61K 31/695; A61F 2/00

(52) U.S. Cl. CPC A61K 47/186; A61K 47/6923; A61F 2002/0086; A61K 31/695; A61F 2/0077; A61K 31/14

(57) ABSTRACT

Anti-microbial active particles, compositions and uses for inhibiting bacterial growth on surfaces or devices are described, with methods of making.

### FIELD OF THE INVENTION

This invention relates to anti-microbial active particles, compositions and uses for inhibiting bacterial growth on surfaces or devices. This invention further provides methods of making such anti-microbial active particles.

### BACKGROUND OF THE INVENTION

The overwhelming diversity of bacteria in one individual's skin, gastro intestinal tract and oral cavity is well documented, demonstrating a complex ecosystem anatomically and dynamically in which poly-microbial biofilms are the norm.

Biofilms formed on tissues outside and inside the organism are the major cause of infectious diseases. For example in the oral cavity, biofilm formed on dental hard or soft tissue are the major cause of caries and periodontal disease (Sbordone L., Bortolaia C., Clin Oral Investig 2003; 7:181-8). Bacterial biofilm forms on both natural and artificial surfaces.

Special attention is paid in recent years to artificial surfaces contacting organisms, as these surfaces lack the epithelial shedding, a major natural mechanism to combat biofilms, thus biofilm accumulation is becoming a major source of medical problems that may result in life threatening complications. Two major factors influence the susceptibility of a surface to accumulate bacteria: surface roughness and the surface-free energy which is a property of the material used. Surface roughness has a higher influence on the adhesion of bacteria than surface-free energy. In this context, artificial restorative materials typically have a higher surface roughness than natural surfaces, and therefore are more prone to bacterial accumulation. Therefore, the development of new materials that diminishes biofilm formation is a critical topic.

The ultimate goal of the development of materials with antibiofilm properties is to improve health and reduce disease occurrence. None of the existing medical devices can guarantee immediate and comprehensive elimination of biofilm or prevention of secondary, infection.

For example, in order to sustain the oral defense, dental materials with the following antibiofilm properties are sought after: (1) inhibition of initial binding of microorganisms (2) preventing biofilm growth, (3) affecting microbial metabolism in the biofilm, (4) killing biofilm bacteria, and (5) detaching biofilm (Busscher H J, Rinastiti M, Siswomihardjo W, van der Mei H C., J Dent Res. 2010; 89:657-65; Marsh P D. J Dent, 2010; 38).

Resin-based composites are complex dental materials that consist of a hydrophobic resin matrix and less hydrophobic filler particles, which implies that a resin-based composite surface is never a homogeneous interface but rather one that produces matrix-rich and filler-poor areas, as well as matrix-poor and filler-rich areas (Ionescu A, Wutscher E, Brambilla E, Schneider-Feyrer S, Cfiessibl F J, Hahne'S.; Eur J Oral Sci 2012; 120:458-65).

Biofilms on composites can cause surface deterioration. Polishing, as well as differences in the composition of the resin-based composite, may have an impact on biofilm formation on the resin-based composite surface (Ono M. et al., Dent Maier J, 2007; 26:613-22). Surface degradation of resin composites driven by polishing leads to increased roughness, changes in micro hardness, and filler particle exposure upon exposure to biofilms in vitro. Furthermore, biofilms on composites can cause surface deterioration. There still remains a need for anti-microbial active materials and it would be advantageous to have an extended variety of anti-microbial active materials which are cost-effective, non-toxic and easy to apply to contaminated surfaces and devices, especially in dental products.

### SUMMARY OF THE INVENTION

This invention provides anti-microbial active functionalized particles, which can be coated on a surface, embedded in a matrix or embedded in raw materials to form compositions demonstrating a broad spectrum of anti-microbial activity. The compositions of the invention are preferably formulated for topical, on mucosal surfaces, skin surfaces, dental surfaces and/or wounds (chronic and acute) administration. The anti-microbial particles prevent the formation of biofilm on surfaces and devices and treat, break down or kill biofilm or bacteria within. Furthermore, this invention provides versatile and cost-effective methodology for the preparation of the anti-microbial active particles.

This invention is based on the surprising discovery that particles comprising an inorganic or organic inert core, and oligomeric or polymeric anti-microbial active group chemically bound to the core directly or via linker—at a surface density of at least one anti-microbial active group per 10 sq. nm, show a broad spectrum of anti-microbial activity when applied to or incorporated onto surfaces and devices on which the growth of such microbes may otherwise naturally take place. Such anti-microbial activity thus prevents biofilm formation and may treat, break down and/or kill biofilm or bacteria within. In some embodiments, the particles generally include an inert core which can be made of an organic polymeric material or inorganic materials, as described herein and an anti-microbial active group. It was found that particles of this invention have high thermal stability.

In some embodiments, this invention provides an anti-microbial active particle comprising:

(i) an inorganic or organic core; and

(ii) polymeric or oligomeric anti-microbial active unit chemically bound to the core directly or indirectly (via a third linker) to the core;

wherein the polymeric or oligomeric anti-microbial active unit comprises more than one monomeric unit comprising an anti-microbial active group; and

wherein the number of the anti-microbial active groups per each anti-microbial active unit is between 1-200.

In one embodiment, the anti-microbially particle is represented by structure (I):

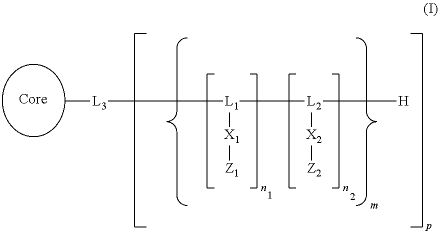

wherein
the core is an organic polymer or an inorganic material;
L₁ is a first linker or a bond;
L₂ is a second linker;
L₃ is a third linker or a bond;

### Z₁ is

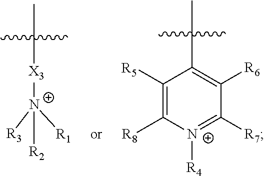

### Z₂ is

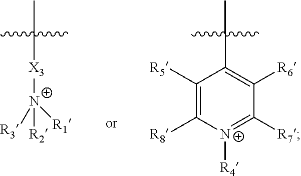

R₁ and R₁′ are each independently methyl, CF₃, perhaloalkyl, aryl, benzyl, 2,2-disubstituted C₃-C₂₀alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ and R₂′ are each independently methyl, CF₃, perhaloalkyl, aryl, benzyl, 2,2-disubstituted C₃-C₂₀alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ and R₃′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₄ and R₄′ are each independently methyl, CF₃, perhaloalkyl, aryl, benzyl, 2,2-disubstituted C₃-C₂₀alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ and R₅′ are each independently H, alkyl, terpenoid moiety, cycloalkyl, aryl, heterocycle, a conjugated alkyl, alkenyl, alkynyl or any combination thereof;
R₆ and R₆′ are each independently H, alkyl, terpenoid moiety, cycloalkyl, aryl, heterocycle, a conjugated alkyl, alkenyl, alkynyl or any combination thereof;
R₇ and R₇′ are each independently H, alkyl, terpenoid moiety, cycloalkyl, aryl, heterocycle, a conjugated alkyl, alkenyl, alkynyl or any combination thereof;
R₈ and R₈′ are each independently H, alkyl, terpenoid moiety, cycloalkyl, aryl, heterocycle, a conjugated alkyl, alkenyl, alkynyl or any combination thereof;
X₁ and X₂ are each independently a bond, alkylene, arylene, alkenylene, alkynylene or any combination thereof;
X₃ and X₄ are each independently a bond, —O—C(═O)—, methylene, —O—C(═O)—CH₂— 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof;
R is alkyl, aryl, cycloalkyl, heterocycle, or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, provided that Z₁ or Z₂ comprises an ammonium nitrogen (not pyridinium)—in each of the anti-microbial active units only one moiety on the ammonium may have beta hydrogens available for hofmann elimination. In another embodiment, provided that Z₁ or Z₂ comprises an ammonium nitrogen (not pyridinium)—in each of the anti-microbial active units two moieties on the ammonium may have beta hydrogens available for hofmann elimination.

In another embodiment, the anti-microbially particle is represented by structure (Ia):

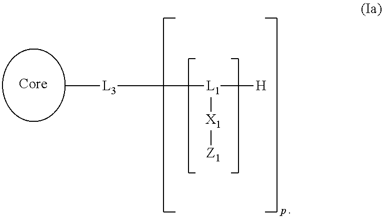

wherein
the core is an organic polymer or an inorganic material;
L₁ is a first linker or a bond;
L₃ is a third linker or a bond;

### Z₁ is

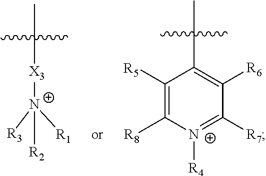

R₁ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ is methyl, CF₃, perhaloalkyl, 2,2-disubstituted C₃-C₁₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₄ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ is H, alkyl, terpenoid moiety, cycloalkyl, aryl, heterocycle, a conjugated alkyl, alkenyl, alkynyl or any combination thereof;
R₆ is H, alkyl, terpenoid moiety, cycloalkyl, aryl, heterocycle, a conjugated alkyl, alkenyl, alkynyl or any combination thereof;
R₇ is H, alkyl, terpenoid moiety, cycloalkyl, aryl, heterocycle, a conjugated alkyl, alkenyl, alkynyl or any combination thereof;
R₈ is H, alkyl, terpenoid moiety, cycloalkyl, aryl, heterocycle, a conjugated alkyl, alkenyl, alkynyl or any combination thereof;
X₁ is a bond, alkylene, arylene, alkenylene, alkynylene or any combination thereof;
X₃ is a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof;
R is alkyl, aryl, cycloalkyl, heterocycle or any combination thereof; and
p defines the number of anti-microbial active unit per one sq nm (nm²) of the core surface, wherein said density is of between 0.01-30 anti-microbial units per one sq nm (nm²) of the core surface of the particle.

In another embodiment, provided that Z₁ comprises an ammonium nitrogen (not pyridinium)—in each of the anti-microbial active units only one moiety on the ammonium may have beta hydrogens available for hofmann elimination. In another embodiment, provided that Z₁ or Z₂ comprises an ammonium nitrogen (not pyridinium)—in each of the anti-microbial active units two moieties on the ammonium may have beta hydrogens available for hofmann elimination.

In another embodiment, the anti-microbially particle is represented by structure (II):

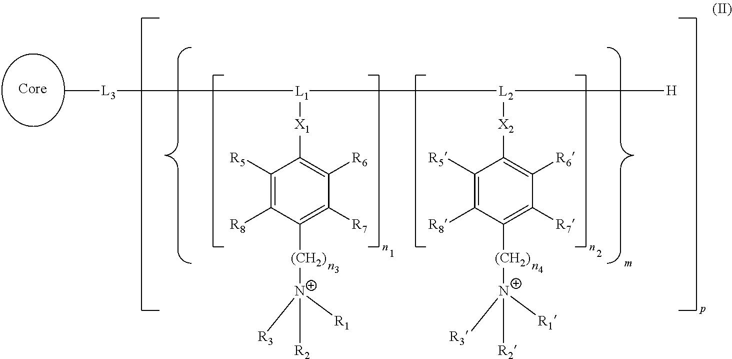

wherein
the core is an organic polymer or an inorganic material;
L₁ is a first linker or a bond;
L₂ is a second linker;
L₃ is a third linker or a bond;
R₁ and R₁′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ and R₂′ are each independently methyl, CF₃, perhaloalkyl, aryl, benzyl, 2,2-disubstituted C₃-C₂P alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ and R₃′ are each independently methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₅ and R₅′ are each independently H, alkyl, terpenoid moiety, cycloalkyl, aryl, heterocycle, a conjugated alkyl, alkenyl, alkynyl or any combination thereof;
R₆ and R₆′ are each independently H, alkyl, terpenoid moiety, cycloalkyl, aryl, heterocycle, a conjugated alkyl, alkenyl, alkynyl or any combination thereof;
R₇ and R₇′ are each independently H, alkyl, terpenoid moiety, cycloalkyl, aryl, heterocycle, a conjugated alkyl, alkenyl, alkynyl or any combination thereof;
R₈ and R₈′ are each independently H, alkyl, terpenoid moiety, cycloalkyl, aryl, heterocycle, a conjugated alkyl, alkenyl, alkynyl or any combination thereof;
X₁ and X₂ are each independently a bond, alkylene, arylene, alkenylene, alkynylene or any combination thereof;
R is alkyl, aryl, cycloalkyl, heterocycle or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
n₃ and n₄ are each independently 0 or 1;
wherein n₁+n₂≥1; and
m is an integer between 1 to 200 and the repeating unit is the same or different.

In another embodiment, in each of the anti-microbial active units only one moiety on the ammonium may have beta hydrogens available for hofmann elimination.

In another embodiment, the anti-microbially particle is represented by structure (III):

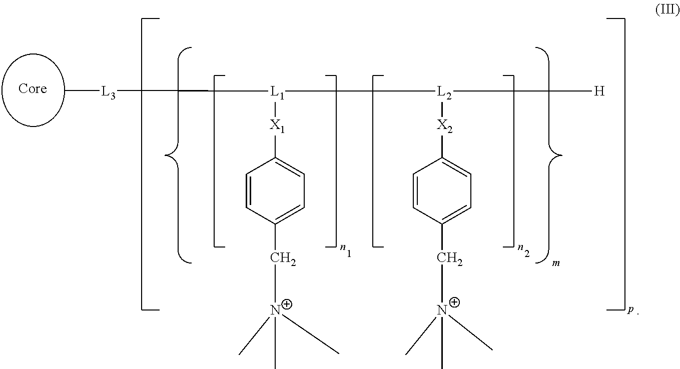

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (IV):

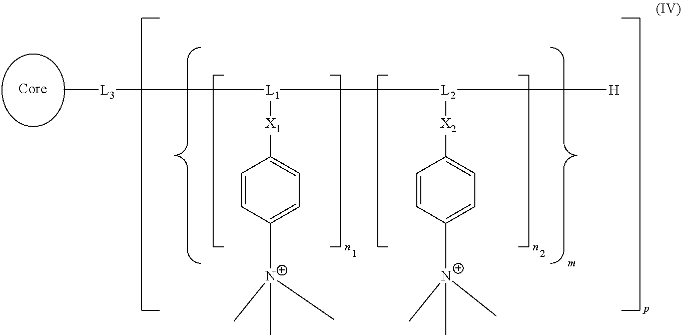

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (V):

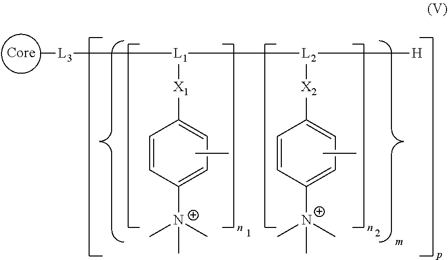

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (VI):

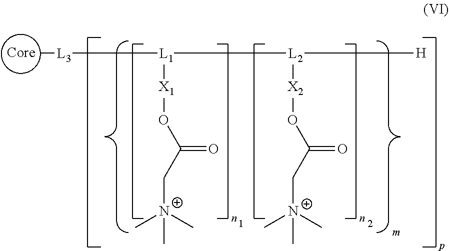

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (VII):

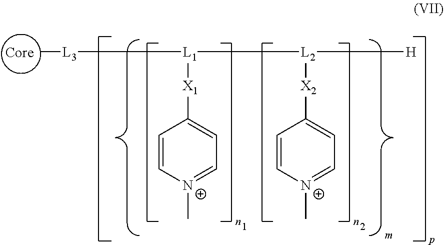

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In one embodiment, this invention provides a composition comprising a polymeric material and an anti-microbial particle as described hereinabove.

In one embodiment, this invention provides a method for inhibiting or preventing biofilm formation or growth comprising administering an anti-microbial particle or a composition as described hereinabove.

In one embodiment, this invention provides a medical device comprising an anti-microbial particle or a composition as described hereinabove.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C depict anti-microbial active particle scheme. FIG. 1A: an oligomeric/polymeric backbone per one anti-microbial active unit; FIG. 1B: a monomeric backbone per one anti-microbial active unit; and FIG. 1C: detailed monomeric unit scheme.

FIG. 2 depicts a representative scheme for the preparation of particles according to this invention wherein the anti-microbial active group is a quaternary ammonium group and the anti-microbial unit has one monomeric unit (a monomeric backbone, as presented in FIG. 1B); the circles represent the organic or inorganic core; R and R′ are each independently methyl, CF₃, perhaloalkyl, aryl, —C(═O)OR, —C(═O)OC(═O)R, —C(═S)OR, —C(═O)SR, —C(═O)—R, —C(═S)—R, 1-alkenyl or 1-alkynyl, where R is alkyl, aryl, cycloalkyl, heterocycle or any combination thereof; R¹ is a methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof and Y is a leaving group such as halogen or sulfonate.

FIGS. 3A-C depicts a representative scheme of three pathways for the preparation of quaternary ammonium salts (QAS) functionalized particle wherein the anti-microbial unit has one monomeric unit (a monomeric backbone, as presented in FIG. 1B); the circles represents organic or inorganic core. FIG. 3A) by alkylation with R₁—Y/R₂—Y to achieve tertiary amine, followed by an benzylation reaction; FIG. 3B) by a similar pathway as in A), done in the reversed order; and FIG. 3C): by reacting a linker functionalized with a leaving group (e.g., Cl or other halogen) with tertiary amine. R₁ and R₂ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —CH₂C(═O)R, —CH₂C(═S)R, —CH₂CF₃, —CH₂NO₂, 1-alkenyl, 1-alkynyl, 2-alkenyl, 2-alkynyl or any combination thereof; where R is alkyl, aryl, cycloalkyl, heterocycle or any combination thereof. Y represents any leaving group, for example Cl, Br or I, or a sulfonate (e.g., mesyl, tosyl).

FIG. 4 depicts schemes of solid support and solution methods for the preparation of particles of this invention wherein the anti-microbial unit has one monomeric unit (a monomeric backbone, as presented in FIG. 1B). The circles represent an organic or inorganic core. Q¹, Q² and Q³ are independently selected from the group consisting of ethoxy, methoxy, methyl, ethyl, hydrogen, sulfonate and halide, wherein at least one of Q¹, Q² and Q³ is a leaving group selected from ethoxy, methoxy, sulfonate (e.g., mesyl, tosyl) and halide. For the sake of clarity the scheme presents a case where Q¹, Q² and Q³ represent leaving groups; Q⁴ represents an anti-microbial group; W is selected from the group consisting of arylene-NH₂, benzylene-NH₂, halide, sulfonate and hydroxyl; and n is an integer between 1 and 16.

FIG. 5 depicts a representative scheme for the preparation of particles according to this invention by a solid support method, wherein the anti-microbial unit has an oligomeric or polymeric backbone (more than one monomeric unit). The circles represent a core. The starting material is a core terminated on the surface with hydroxyl groups; Q¹⁰¹, Q¹⁰² and Q¹⁰³ are each independently alkoxy, alkyl or aryl; LG is Cl, Br, I, mesylate, tosylate or triflate; Hal is Cl, Br or I; q, q¹, q² and q³ are each independently an integer between 0-16; Riand R₂ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof; and R₃ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof.

FIGS. 6A-6C depict self-polymerization of trialkoxysilane linker. FIG. 8A: self-polymerization of trialkoxysilane linker via solid support method; FIG. 8B: self-polymerization of trialkoxysilane linker in solution; and FIG. 8C: comparison of polymerization of the silane groups versus simple silanization.

FIG. 7 depicts a representative scheme for the preparation of particles according to this invention in a solution method, wherein the anti-microbial unit has more than one monomeric unit (i.e has an oligomeric or polymeric backbone). The circles represent a core. The starting material is a core terminated on the surface with hydroxyl groups; Q¹⁰¹, Q¹⁰² and Q¹⁰³ and independently alkoxy, alkyl or aryl; LG is Cl, Br, I, mesylate, tosylate or triflate; Hal is Cl, Br or I; q, q¹, q² and q³ are independently an integer between 0-16; R¹ and R₂ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof; and R₃ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof.

FIG. 8 depicts a scheme for the preparation of silica based anti-microbial particles according to this invention comprising dimethylbenzylammonium as the anti-microbial active group, in a solid support method, wherein the anti-microbial unit has more than one monomeric unit (i.e has an oligomeric or polymeric backbone).

FIG. 9 depicts a scheme for the preparation of silica based anti-microbial particles according to this invention comprising dimethylbenzylammonium as the anti-microbial active group, in a solution method, wherein the anti-microbial unit has more than one monomeric unit (i.e has an oligomeric or polymeric backbone).

FIG. 10 depicts results of differential scanning calorimetry experiments performed for three samples: (2QA POSS)—linker is aliphatic chain, hydrophobic group is octyl; (2QA BP)—linker is aliphatic chain, hydrophobic group is benzyl; and (QA BP)—linker is aliphatic chain with hydroxy onto β-carbon and the hydrophobic group is benzyl.

FIGS. 11A-11C depict pictures of agar plates with E. faecalis suspension in brain heart infusion (BHT) following incubation with antibacterial particles according to this invention and as detailed in Example 2. FIG. 11A: polypropylene (PP) rods (control) at left and particles at 5% w/w in PP at right; FIG. 11B: particles at 10% w/w in PP; and FIG. 11C: polypropylene (PP) rods (control) at left and particles at 10% w/w in PP at right.

FIGS. 12A-12B depict results following direct contact test (DCT) with anti bacterial particles according to this invention and as detailed in Example 3. FIG. 12A: bacteria growth curve; and FIG. 12B: calibration curve prepared for the E. faecalis suspension used in the DCT where the name of each curve (“1”, “1.3E-01” . . . ) corresponds to its relative concentration.

It will be appreciated that for simplicity and clarity of illustration, elements shown in the Figures have not necessarily been drawn to scale. For example, the dimensions of some of the elements may be exaggerated relative to other elements for clarity. Further, where considered appropriate, reference numerals may be repeated among the Figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention. In some embodiments, this invention provides an anti-microbial active particle comprising:

(i) an inorganic or organic core; and

(ii) polymeric or oligomeric anti-microbial active unit chemically bound to the core directly or indirectly (via a third linker) to the core;

wherein the polymeric or oligomeric anti-microbial active unit comprises more than one monomeric unit comprising an anti-microbial active group; and

wherein the number of the anti-microbial active groups per each anti-microbial active unit is between 1-200.

In some embodiments, the anti-microbial particles have high thermal stability. Without being bound by any mechanism or theory, it is suggested that the high stability stems from lack of available beta (β) hydrogens on the ammonium or a low number thereof, thus reducing the possibility of having a hofmann elimination which in turn gives rise to reduced thermal stability.

In some embodiments, the anti-microbial particles comprise (i) an inorganic or organic core; and (ii) an anti-microbial active part chemically bound to the core. In one embodiment, the anti-microbial active part comprises one monomeric unit. In one embodiment, the anti-microbial active part comprises more than one monomeric unit. In another embodiment, the anti-microbial active part with the more than one monomeric unit comprises more than one linker. In another embodiment, the anti-microbial active unit has between 2-200 monomeric units. In another embodiment, the anti-microbial active unit has between 2-5 monomeric units. In another embodiment, the anti-microbial active unit has between 5-10 monomeric units. In another embodiment, the anti-microbial active unit has between 10-20 monomeric units. In another embodiment, the anti-microbial active unit has between 20-50 monomeric units. In another embodiment, the anti-microbial active unit has between 50-100 monomeric units. In another embodiment, the anti-microbial active unit has between 100-200 monomeric units.

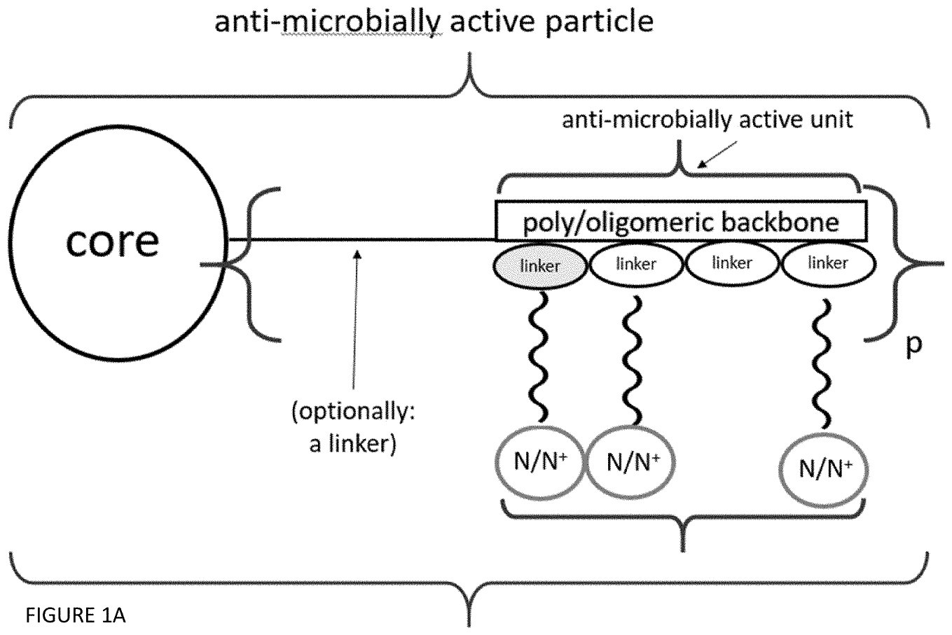

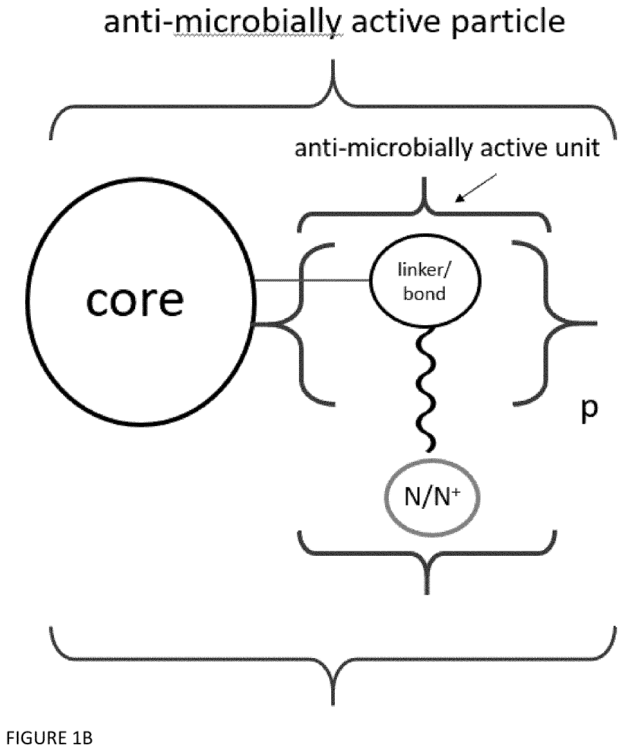

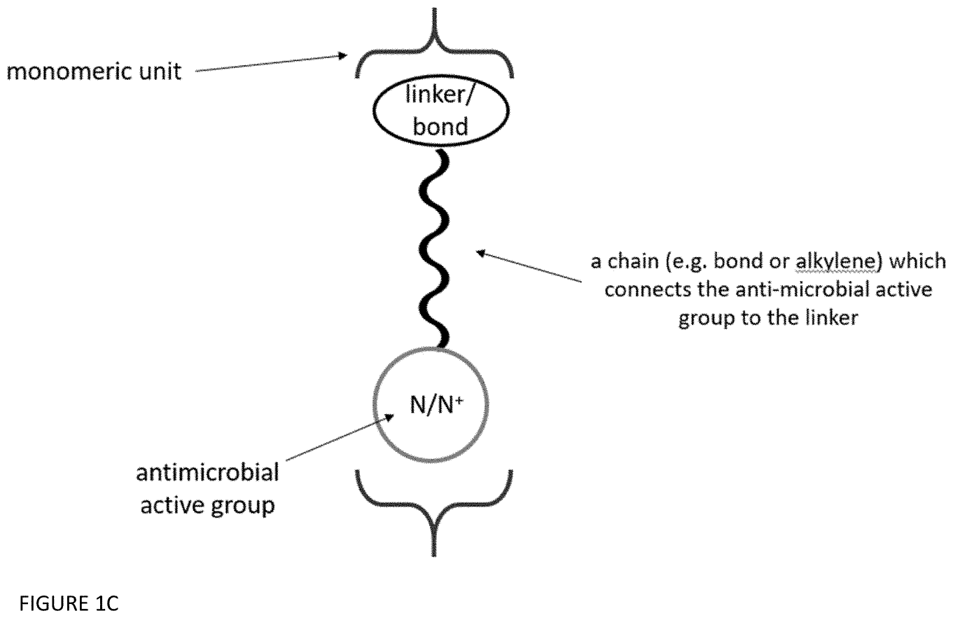

In one embodiment, the anti-microbial active unit comprises more than one monomeric unit. In another embodiment, the monomeric units are connected to each other via a first linker, a second linker or both. In another embodiment, each monomeric unit comprises an anti-microbial active group. In another embodiment, an anti-microbial active unit comprises at least one anti-microbial active group. In another embodiment, an anti-microbial active unit comprises at least two anti-microbial active groups. In another embodiment, FIGS. 1A, 1B and 1C illustrate schematically the anti-microbial active particles of this invention (FIG. 1A: more than one monomer; FIG. 1B: one monomeric unit and FIG. 1C: detailed scheme of one monomer).

In one embodiment, the anti-microbially particle is represented by structure (I):

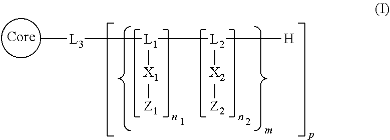

wherein
the core is an organic polymer or an inorganic material;
L₁ is a first linker or a bond;
L₂ is a second linker;
L₃ is a third linker or a bond;

### Z₁ is

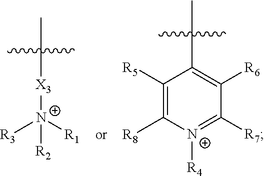

### Z₂ is

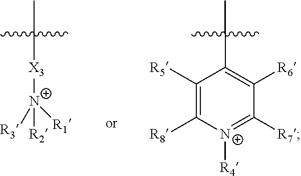

R₁ and R₁′ are each independently methyl, CF₃, perhaloalkyl, aryl, benzyl, 2,2-disubstituted C₃-C₂₀alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ and R₂′ are each independently methyl, CF₃, perhaloalkyl, aryl, benzyl, 2,2-disubstituted C₃-C₂₀alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ and R₃′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₄ and R₄′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ and R₅′ are each independently H, alkyl, terpenoid moiety, cycloalkyl, aryl, heterocycle, a conjugated alkyl, alkenyl, alkynyl or any combination thereof;
R₆ and R₆′ are each independently H, alkyl, terpenoid moiety, cycloalkyl, aryl, heterocycle, a conjugated alkyl, alkenyl, alkynyl or any combination thereof;
R₇ and R₇′ are each independently H, alkyl, terpenoid moiety, cycloalkyl, aryl, heterocycle, a conjugated alkyl, alkenyl, alkynyl or any combination thereof;
R₈ and R₈′ are each independently H, alkyl, terpenoid moiety, cycloalkyl, aryl, heterocycle, a conjugated alkyl, alkenyl, alkynyl or any combination thereof;
X₁ and X₂ are each independently a bond, alkylene, arylene, alkenylene, alkynylene or any combination thereof;
X₃ and X₄ are each independently a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof;
R is alkyl, aryl, cycloalkyl, heterocycle or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, provided that Z₁ or Z₂ comprises an ammonium nitrogen (not pyridinium)—in each of the anti-microbial active units only one moiety on the ammonium may have beta hydrogens available for hofmann elimination. In another embodiment, provided that Z₁ or Z₂ comprises an ammonium nitrogen (not pyridinium)—in each of the anti-microbial active units two moieties on the ammonium may have beta hydrogens available for hofmann elimination. In another embodiment, beta hydrogens available for hofmann elimination are those which are found on beta (compared to the ammonium nitrogen) aliphatic carbon and can be eliminated to release an olefin and a tertiary amine.

In another embodiment, the anti-microbially particle is represented by structure (Ia):

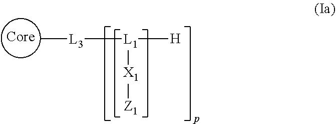

wherein
the core is an organic polymer or an inorganic material;
L₁ is a first linker or a bond;
L₃ is a third linker or a bond;

### Z₁ is

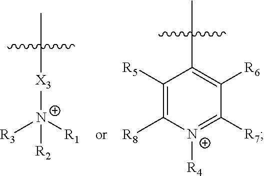

R₁ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₄ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ is H, alkyl, terpenoid moiety, cycloalkyl, aryl, heterocycle, a conjugated alkyl, alkenyl, alkynyl or any combination thereof;
R₆ is H, alkyl, terpenoid moiety, cycloalkyl, aryl, heterocycle, a conjugated alkyl, alkenyl, alkynyl or any combination thereof;
R₇ is H, alkyl, terpenoid moiety, cycloalkyl, aryl, heterocycle, a conjugated alkyl, alkenyl, alkynyl or any combination thereof;
R₈ is H, alkyl, terpenoid moiety, cycloalkyl, aryl, heterocycle, a conjugated alkyl, alkenyl, alkynyl or any combination thereof;
X₁ is a bond, alkylene, arylene, alkenylene, alkynylene or any combination thereof;
X₃ is a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof;
R is alkyl, aryl, cycloalkyl, heterocycle or any combination thereof; and
p defines the number of anti-microbial active unit per one sq nm (nm²) of the core surface, wherein said density is of between 0.01-30 anti-microbial units per one sq nm (nm²) of the core surface of the particle.

In another embodiment, provided that Z₁ comprises an ammonium nitrogen (not pyridinium)—in each of the anti-microbial active units only one moiety on the ammonium may have beta hydrogens available for hofmann elimination. In another embodiment, provided that Z₁ or Z₂ comprises an ammonium nitrogen (not pyridinium)—in each of the anti-microbial active units two moieties on the ammonium may have beta hydrogens available for hofmann elimination.

In another embodiment, the anti-microbially particle is represented by structure (II):

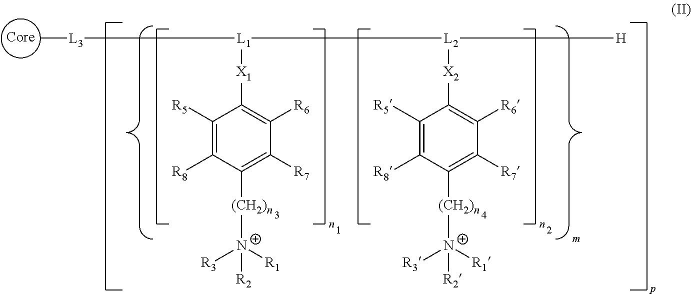

wherein
the core is an organic polymer or an inorganic material;
L₁ is a first linker or a bond;
L₂ is a second linker;
L₃ is a third linker or a bond;
R₁ and R₁′ are each independently methyl, CF₃, perhaloalkyl, aryl, benzyl, 2,2-disubstituted C₃-C₂P alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ and R₂′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ and R₃′ are each independently methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₅ and R₅′ are each independently H, alkyl, terpenoid moiety, cycloalkyl, aryl, heterocycle, a conjugated alkyl, alkenyl, alkynyl or any combination thereof;
R₆ and R₆′ are each independently H, alkyl, terpenoid moiety, cycloalkyl, aryl, heterocycle, a conjugated alkyl, alkenyl, alkynyl or any combination thereof;
R₇ and R₇′ are each independently H, alkyl, terpenoid moiety, cycloalkyl, aryl, heterocycle, a conjugated alkyl, alkenyl, alkynyl or any combination thereof;
R₈ and R₈′ are each independently H, alkyl, terpenoid moiety, cycloalkyl, aryl, heterocycle, a conjugated alkyl, alkenyl, alkynyl or any combination thereof;
X₁ and X₂ are each independently a bond, alkylene, arylene, alkenylene, alkynylene or any combination thereof;
R is alkyl, aryl, cycloalkyl, heterocycle or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
n₃ and n₄ are each independently 0 or 1;
wherein n₁+n₂≥1; and
m is an integer between 1 to 200 and the repeating unit is the same or different.

In another embodiment, in each of the anti-microbial active units only one moiety on the ammonium may have beta hydrogens available for hofmann elimination.

In another embodiment, the anti-microbially particle is represented by structure (III):

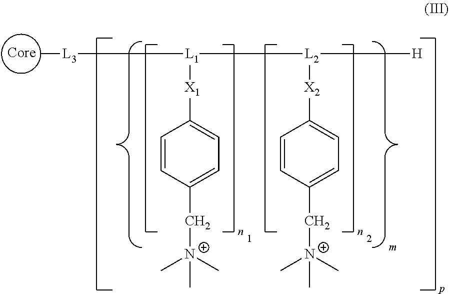

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (IV):

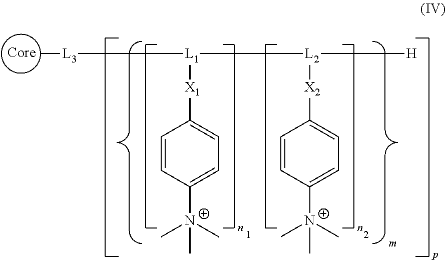

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (V):

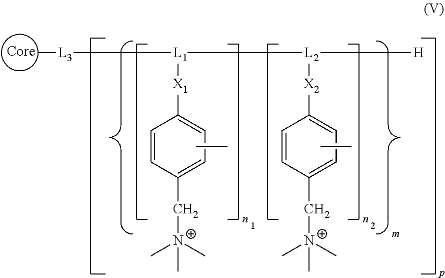

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In another embodiment, the anti-microbially particle is represented by structure (VI):

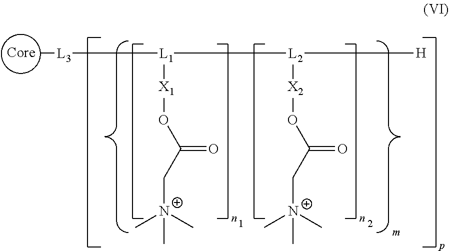

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n2≥1; and
m is an integer between 1 to 200 and the repeating unit is the same or different.

In another embodiment, the anti-microbially particle is represented by structure (VII):

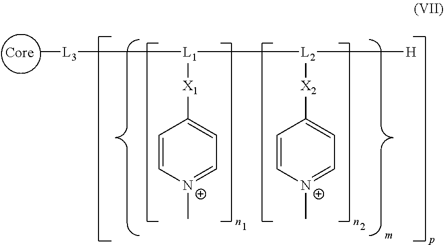

wherein
the core is an organic polymer or an inorganic material;
L₁ is a first linker or a bond;
L₂ is a second linker;
L₃ is a third linker or a bond;
X₁ and X₂ are each independently a bond, alkylene, arylene, alkenylene, alkynylene or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

In some embodiments, the term “anti-microbial active group” and the term “monomeric anti-microbial active group” refer to the same and comprise a quaternary ammonium and/or a pyridinium, as represented by the following formulas:

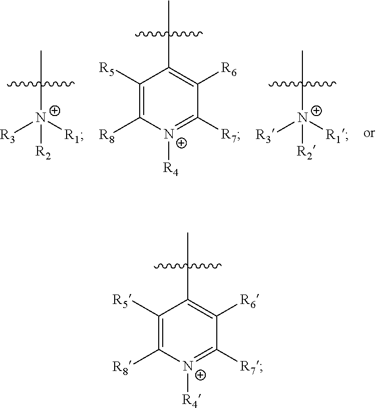

wherein:
R₁-R₈ and R₁′-R₈′ are as described hereinabove.

In another embodiment, the number of the anti-microbial active groups per each anti-microbial active unit is at least two, i.e. n₁+n₂≥2 and m≥1. In another embodiment, the number of the anti-microbial active groups per each anti-microbial active unit is one, i.e. n₁+n₂=1 and m=1.

In another embodiment, the particles of structure (Ia) comprise one monomeric unit per one anti-microbial active unit. In another embodiment, the particles of structures (I) and (II) to (VII) comprise one or more than one anti-microbial active group per one anti-microbial active unit.

The anti-microbial active groups of this invention are chemically bound to the core at a surface density of at least one anti-microbial active group per 10 sq. nm of the core surface. In another embodiment at least 1 anti-microbial group per 1 sq nm of the core surface. In another embodiment between 0.001-300 anti-microbial groups per sq nm of the core surface. In another embodiment between 0.001-250 anti-microbial groups per sq nm of the core surface. In another embodiment between 0.001-200 anti-microbial groups per sq nm of the core surface. In another embodiment between 0.001-150 anti-microbial groups per sq nm of the core surface. In another embodiment between 0.001-100 anti-microbial groups per sq nm of the core surface. In another embodiment between 0.001-50 anti-microbial groups per sq nm of the core surface. In another embodiment between 0.001-20 anti-microbial groups per sq nm of the core surface. In another embodiment between 0.001-17 anti-microbial groups per sq nm of the core surface. In another embodiment between 0.001-15 anti-microbial groups per sq nm of the core surface. In another embodiment between 0.001-10 anti-microbial groups per sq nm of the core surface. In another embodiment between 0.001-4 anti-microbial groups per sq nm of the core surface. In another embodiment between 0.001-1 anti-microbial groups per sq nm of the core surface. In another embodiment between 50-100 anti-microbial groups per sq nm of the core surface. In another embodiment between 100-150 anti-microbial groups per sq nm of the core surface. In another embodiment between 150-200 anti-microbial groups per sq nm of the core surface. In another embodiment between 200-250 anti-microbial groups per sq nm of the core surface. In another embodiment between 250-300 anti-microbial groups per sq nm of the core surface. In another embodiment between 1-4 anti-microbial groups per sq nm of the core surface. In another embodiment between 1-6 anti-microbial groups per sq nm of the core surface. In another embodiment between 1-20 anti-microbial groups per sq nm of the core surface. In another embodiment between 1-10 anti-microbial groups per sq nm of the core surface. In another embodiment between 1-15 anti-microbial groups per sq nm of the core surface.

In some embodiments, the number of the anti-microbial active groups [(n₁+n₂)×m] per each anti-microbial active unit is between 1-200. In another embodiment, the number of the anti-microbial active groups per each anti-microbial active unit is between 1-150. In another embodiment, the number of the anti-microbial active groups per each anti-microbial active unit is between 1-100. In another embodiment, the number of the anti-microbial active groups per each anti-microbial active unit is between 1-50. In another embodiment, the number of the anti-microbial active groups per each anti-microbial active unit is between 1-30. In another embodiment, the number of the anti-microbial active groups per each anti-microbial active unit is between 1-20. In another embodiment, the number of the anti-microbial active groups per each anti-microbial active unit is between 1-10. In another embodiment, the number of the anti-microbial active groups per each anti-microbial active unit is between 50-100. In another embodiment, the number of the anti-microbial active groups per each anti-microbial active unit is between 100-150. In another embodiment, the number of the anti-microbial active unit per each anti-microbial active unit is between 150-200.

In some embodiments, the number of the monomeric units per each anti-microbial active unit is between 1-200. In another embodiment, the number of the monomeric units per each anti-microbial active unit is between 1-150. In another embodiment, the number of the monomeric units per each anti-microbial active unit is between 1-100. In another embodiment, the number of the monomeric units per each anti-microbial active unit is between 1-50. In another embodiment, the number of the monomeric units per each anti-microbial active unit is between 1-30. In another embodiment, the number of monomeric units per each anti-microbial active unit is between 1-20. In another embodiment, the number of the monomeric units per each anti-microbial active unit is between 1-10. In another embodiment, the number of the monomeric units per each anti-microbial active unit is between 50-100. In another embodiment, the number of the monomeric units per each anti-microbial active unit is between 100-150. In another embodiment, the number of the monomeric units per each anti-microbial active unit is between 150-200.

In another embodiment, the particle of structures (I) to (VII) has an inorganic core. In another embodiment, the particle of structure (I) to (VII) has an organic core. In another embodiment, the organic core is a polymeric organic core. In another embodiment, the core is inert.

In one embodiment, Z₁ is

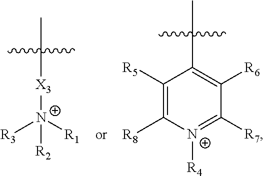

wherein X₃ and R₁-R₈ are as described hereinbelow. Each possibility represents a separate embodiment of this invention.

In one embodiment, Z₂ is

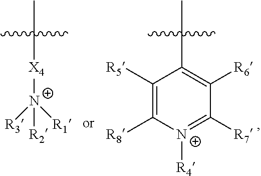

wherein X₄ and R₁′-R₈′ are as described hereinbelow. Each possibility represents a separate embodiment of this invention.

In one embodiment, R₁ and/or RC, R₂ and/or R₂′ and R₄ and/or R₄′ are the same or different and are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, wherein R is described hereinbelow. Each possibility represents a separate embodiment of this invention.

In one embodiment, R₃ and R₃′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;

Each possibility represents a separate embodiment of this invention.

In one embodiment, R₅ and/or R₅′, R₆ and/or R₆′, R₇ and/or R₇′ and R₈ and/or R₈′ are the same or different and are independently H, alkyl, terpenoid moiety, cycloalkyl, aryl, heterocycle, a conjugated alkyl, alkenyl, alkynyl or any combination thereof. Each possibility represents a separate embodiment of this invention.

In one embodiment, X₁ and/or X₂ are the same or different and are independently a bond, alkylene, arylene, alkenylene, alkynylene or any combination thereof. Each possibility represents a separate embodiment of this invention.

In one embodiment, X₃ and X₄ are each independently a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof. Each possibility represents a separate embodiment of this invention.

In one embodiment, R is alkyl, aryl, cycloalkyl, heterocycle or any combination thereof. Each possibility represents a separate embodiment of this invention.

In another embodiment R₁ and R₁′ are the same. In another embodiment R₂ and R₂′ are the same. In another embodiment R₃ and R₃′ are the same. In another embodiment R₄ and R₄′ are the same. In another embodiment R₅ and R₅′ are the same. In another embodiment R₆ and R₆′ are the same. In another embodiment R₇ and R₇′ are the same. In another embodiment R₈ and R₈′ are the same. In another embodiment X₁ and X₂ are the same. In another embodiment X₃ and X₄ are the same. In another embodiment R₁ and R₁′ are different. In another embodiment R₂ and R₂′ are different. In another embodiment R₃ and R₃′ are different. In another embodiment R₄ and R₄′ are different. In another embodiment R₅ and R₅′ are different. In another embodiment R₆ and R₆′ are different. In another embodiment R₇ and R₇′ are different. In another embodiment R₈ and R₈′ are different. In another embodiment X₁ and X₂ are different. In another embodiment X₃ and X₄ are different.

As used herein, the term “alkyl” or “alkylene” refer to any linear- or branched-chain alkyl group containing up to about 24 carbons unless otherwise specified. In one embodiment, an alkyl includes C₁-C₃ carbons. In one embodiment, an alkyl includes C₁-C₄ carbons. In one embodiment, an alkyl includes C₁-C₅ carbons. In another embodiment, an alkyl includes C₁-C₆ carbons. In another embodiment, an alkyl includes C₁-C₈ carbons. In another embodiment, an alkyl includes C₁-C₁₀ carbons. In another embodiment, an alkyl includes C₁-C₁₂ carbons. In another embodiment, an alkyl includes C₄-C₈ carbons. In another embodiment, an alkyl includes C₄-C₁₀ carbons. In another embodiment, an alkyl include C₄-C₁₈ carbons. In another embodiment, an alkyl include C₄-C₂₄ carbons. In another embodiment, an alkyl includes C₁-C₁₈ carbons. In another embodiment, an alkyl includes C₂-C₁₈ carbons. In another embodiment, branched alkyl is an alkyl substituted by alkyl side chains of 1 to 5 carbons. In one embodiment, the alkyl group may be unsubstituted. In another embodiment, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the alkyl is a 2,2-disubstituted C₃-C₂₀ alkyl. The term “2,2-disubstituted C₃-C₂₀ alkyl” refers to alkyl as described herein, having between 3 and 20 carbons and is substituted thrice at the second carbon (from the connection point) with halogen, haloalkyl, alkyl, alkoxy, carbonyl, amido, alkylamido, dialkylamido, cyano, nitro, CO₂H, amino, alkylamino, dialkylamino, carboxyl, thio and/or thioalkyl, where such substitutions can be the same or different; or alternatively it is substituted once at the second carbon with oxo (═O) or with other double bond to an element (e.g. S) or a moiety (e.g. vinylic carbon or NH) and it's further substituted with a substitutent selected from the above list of the first possibility; in all cases—no hydrogen is available for abstraction at this second carbon position (i.e. no hydrogens are found at this position, only non-hydrogen substituents). Non-limiting examples of 2,2-disubstituted C₃-C₂₀ alkyl include neopentyl (—CH₂—C(CH₃)₃, —CH₂—C(CH₃)₂—CH₂CH₃, CH₂—CF₂CH₃ and —CH₂C(═O)CH₃. In another embodiment, the alkyl is a 2,2-disubstituted C₃-C₅ alkyl. In another embodiment, the alkyl is a 2,2-disubstituted C₃-C₁₀ alkyl. In another embodiment, the alkyl is a 2,2-disubstituted C₃-C₁₂ alkyl. In another embodiment, the alkyl is a 2,2-disubstituted C₃-C₁₈ alkyl. The terms “2,2-disubstituted C₃-C₅ alkyl”, “2,2-disubstituted C₃-C₁₀ alkyl”, “2,2-disubstituted C₃-C₁₂ alkyl” and “2,2-disubstituted C₃-C₁₈ alkyl” refer to similar moiety as “2,2-disubstituted C₃-C₂₀ alkyl” but with C₃-C₈, C₃-C₁₀, C₃-C₁₂ and C₃-C₁₈ alkyl, respectively. In another embodiment, alkylene is a 2,2-disubstituted C₂-C₂) alkylene. The term “2,2-disubstituted C₂-C₂₀ alkylene” refers to similar moiety as “2,2-disubstituted C₃-C₂₀ alkyl” but with alkylene as described herein which has between 2 and 20 carbons. Non-limiting examples of 2,2-disubstituted C₂-C₂₀ alkylene include neopentylene (—CH₂—C(CH₃)₂—CH₂—, —CH₂—C(CH₃)₂—CH₂CH₂—, —CH₂—CF₂CH₂— and —CH₂C(═O)CH₂—. In another embodiment, the alkylene is a 2,2-disubstituted C₂-C₅ alkylene. In another embodiment, the alkylene is a 2,2-disubstituted C₂-C₁₀ alkylene. In another embodiment, the alkylene is a 2,2-disubstituted C₂-C₁₂ alkylene. In another embodiment, the alkylene is a 2,2-disubstituted C₂-C₁₈ alkylene. The terms “2,2-disubstituted C₂-C₈ alkylene”, “2,2-disubstituted C₂-C₁₀ alkylene”, “2,2-disubstituted C₂-C₁₂ alkylene” and “2,2-disubstituted C₂-C₁₈ alkylene” refer to similar moiety as “2,2-disubstituted C₂-C₂₀ alkylene” but with C₂-C₈, C₂-C₁₀, C₂-C₁₂ and C₂-C₁₈ alkylene, respectively.

In another embodiment, the alkyl is a 2,2,2-trisubstituted ethyl. The term “2,2,2-trisubstituted ethyl” refers to ethyl substituted thrice at the second carbon (from the connection point) with halogen, haloalkyl, alkoxy, carbonyl, amido, alkylamido, dialkylamido, cyano, nitro, CO₂H, amino, alkylamino, dialkylamino, carboxyl, thio and/or thioalkyl, where such substitutions can be the same or different; or alternatively it is substituted once at the second carbon with oxo (═O) or with other double bond to an element (e.g. S) or a moiety (e.g. vinylic carbon or NH) and it's further substituted with a substituent selected from the above list of the first possibility; in all cases—no hydrogen is available for abstraction at this second carbon position (i.e. no hydrogens are found at this position, only non-hydrogen substituents). Non-limiting examples of 2,2,2-trisubstituted ethyl include 2,2,2 trihaloethyl and —CH₂C(═O)—NH₂. In another embodiment hydrophobic alkyl refers to an alkyl having at least four carbons. In another embodiment hydrophobic alkyl refers to a C₄-C₂₄ alkyl. In another embodiment hydrophobic alkyl refers to a C₄-C₅ alkyl. In another embodiment hydrophobic alkyl refers to a C₄ alkyl. In another embodiment hydrophobic alkyl refers to a C₅ alkyl. In another embodiment hydrophobic alkyl refers to a C₆ alkyl. In another embodiment hydrophobic alkyl refers to a C₇ alkyl. In another embodiment hydrophobic alkyl refers to a C₈ alkyl.

As used herein, the term “aryl” refers to any aromatic ring that is directly bonded to another group and can be either substituted or unsubstituted. As used herein, the term “Arylene” refers to the same where it is directly bonded to two groups (i.e. arylene is e.g. phenylene, —C₆H₄). In another embodiment, it can be directly bonded to more than two groups. The aryl or arylene group can be a sole substituent, or it can be a component of a larger substituent, such as in an arylalkyl, arylamino, arylamido, etc. Exemplary aryl (and similarly, arylene) groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkyl or alkoxy, C₁-C₅ linear or branched haloalkyl or haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂. In another embodiment, hydrophobic aryl or arylene refers to aryl or arylene having at least six carbons.

As used herein, the term “benzyl” refers to the —CH₂—C₆H₅ moiety and can be unsubstituted or substituted with the following non-limiting list of substituents: F, Cl, Br, I, C₁-C₅ linear or branched alkyl, C₁-C₅ linear or branched haloalkyl, C₁-C₅ linear or branched alkyl or alkoxy, C₁-C₅ linear or branched haloalkyl or haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂. Similarly, “benzylene” refers to the —CH₂—C₆H₄— moiety and can be unsubstituted or substituted with the substituents described above for the benzyl moiety.

As used herein, the term “haloalkyl” refers to alkyl as described hereinabove and substituted at least once by halide (i.e. F, Cl, Br or I). In one embodiment, all of the alkyl is substituted by halides, i.e. no hydrogens are found in the haloalkyl, and is termed “perhaloalkyl” (e.g. CF₃: perfluoromethyl or CCl₃: perchloromethyl). In one embodiment, only part of the alkyl is substituted by halides (e.g. CH₂CF₃). In another embodiment, non limiting examples of haloalkyls include: CF₃, CCl₃, CH₂CF₃, CF₂CF₃, CCl₂CCl₃ and CI₃.

The term “alkenyl” or “alkenylene” refer to a substance that includes at least two carbon atoms and at least one double bond. The term “1-alkenyl” or “1-alkenylene” refers to the same, where the double bond is on the first carbon (from the connection point). The term “2-alkenyl” or “2-alkenylene” refers to the same, where the double bond is on the second carbon (from the connection point). The term “3-alkenyl” or “3-alkenylene” refers to the same, where the double bond is on the third carbon (from the connection point). In one embodiment, the alkenyl has 2-7 carbon atoms. In another embodiment, the alkenyl has 2-12 carbon atoms. In another embodiment, the alkenyl has 2-10 carbon atoms. In another embodiment, the alkenyl has 3-6 carbon atoms. In another embodiment, the alkenyl has 2-4 carbon atoms. In another embodiment, the alkenyl has 4-8 carbon atoms. In another embodiment hydrophobic alkenyl refers to alkenyl having at least four carbons. In another embodiment hydrophobic alkenyl refers to a C₄-C₈ alkenyl.

The term “alkynyl” or “alkynylene” refers to a substance that includes at least two carbon atoms and at least one triple bond. The term “1-alkynyl” or “1-alkynylene” refers to the same, where the triple bond is on the first carbon (from the connection point). The term “2-alkynyl” or “2-alkynylene” refers to the same, where the triple bond is on the second carbon (from the connection point). The term “3-alkynyl” or “3-alkynylene” refers to the same, where the triple bond is on the third carbon (from the connection point). In one embodiment, the alkynyl has 2-7 carbon atoms. In another embodiment, the alkynyl has 2-12 carbon atoms. In another embodiment, the alkynyl has 2-10 carbon atoms. In another embodiment, the alkynyl has 3-6 carbon atoms. In another embodiment, the alkynyl has 2-4 carbon atoms. In another embodiment, the alkynyl has 3-6 carbon atoms. In another embodiment, the alkynyl has 4-8 carbon atoms. In another embodiment hydrophobic alkynyl refers to alkynyl having at least four carbons. In another embodiment hydrophobic alkynyl refers to a C₄-C₈ alkenyl.

The term “alkoxy” refers in one embodiment to an alky as defined above bonded to an oxygen. Non limiting examples of alkoxy groups include: methoxy, ethoxy and isopropoxy.

A “cycloalkyl” group refers, in one embodiment, to a ring structure comprising carbon atoms as ring atoms, which may be either saturated or unsaturated, substituted or unsubstituted; and is directly bonded to one group (e.g. cyclohexyl-, C₆H₁₁—). In another embodiment the cycloalkyl is a 3-12 membered ring. In another embodiment the cycloalkyl is a 6 membered ring. In another embodiment the cycloalkyl is a 5-7 membered ring. In another embodiment the cycloalkyl is a 3-8 membered ring. In another embodiment, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the cycloalkyl ring may be fused to another saturated or unsaturated cycloalkyl or heterocyclic 3-8 membered ring. In another embodiment, the cycloalkyl ring is a saturated ring. In another embodiment, the cycloalkyl ring is an unsaturated ring. Non-limiting examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc. In another embodiment hydrophobic cycloalkyl refers to a cycloalkyl having at least six carbons. A “cycloalkylene” group refers, in one embodiment, to the same definitions above of “cycloalkyl”, however the cycloalkylene is directly bonded to two groups (e.g. -cyclohexylene-, —C₆H₁₀—). In another embodiment, it is directly bonded to more than two groups.

A “heterocycle” group refers, in one embodiment, to a ring structure comprising in addition to carbon atoms, sulfur, oxygen, nitrogen or any combination thereof, as part of the ring. In another embodiment the heterocycle is a 3-12 membered ring. In another embodiment the heterocycle is a 6 membered ring. In another embodiment the heterocycle is a 5-7 membered ring. In another embodiment the heterocycle is a 3-8 membered ring. In another embodiment, the heterocycle group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the heterocyclic ring is a saturated ring. In another embodiment, the heterocyclic ring is an unsaturated ring. Non limiting examples of a heterocyclic rings comprise pyridine, piperidine, morpholine, piperazine, thiophene, pyrrole, benzodioxole, or indole. In another embodiment hydrophobic heterocyclic group refers to a heterocycle having at least six carbons. In one embodiment, the heterocycle is directly bonded to one group (e.g. pyridinyl,

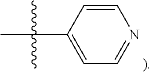

In one embodiment, the heterocycle is directly bonded to two groups (e.g. pyridinylene,

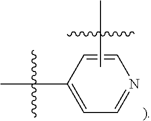

In one embodiment, the heterocycle is directly bonded to more than two groups.

In another embodiment, at least one of R₁, R₂ and R₃ and/or at least one of R₁′, R₂′ and R₃′ of structure (I) is/are hydrophobic.

The term “hydrophobic” refers to an alkyl, alkenyl or alkynyl having at least four carbons, or the term hydrophobic refers to terpenoid, to cycloalkyl, aryl or heterocycle having at least six carbons. Each possibility represents a separate embodiment of this invention

In another embodiment, at least one of R₃, R₅—R₈ and X₃ and/or at least one of R₃′, R₅′-R₈′ and X₄ of structure (I) is a terpenoid. Each possibility represents a separate embodiment of this invention.

In one embodiment “p” of structures (I) to (VII) defines the surface density of the anti-microbial active units per 1 sq nm of the core surface. In another embodiment “p” is, between 0.01-30 anti-microbial active units per 1 sq nm of the core surface. In another embodiment “p” is, between 0.01-20 anti-microbial active units per 1 sq nm of the core surface. In another embodiment “p” is, between 0.01-10 anti-microbial active units per 1 sq nm of the core surface. In another embodiment “p” is, between 0.01-15 anti-microbial active units per 1 sq nm of the core surface. In another embodiment “p” is, between 0.01-5 anti-microbial active units per 1 sq nm of the core surface.

In one embodiment, n₁ of structures (I) and (II) to (VII) is between 0-200. In another embodiment, n₁ is between 0-10. In another embodiment, n₁ is between 10-20. In another embodiment, n₁ is between 20-30. In another embodiment, n₁ is between 30-40. In another embodiment, n₁ is between 40-50. In another embodiment, n₁ is between 50-60. In another embodiment, n₁ is between 60-70. In another embodiment, n₁ is between 70-80. In another embodiment, n₁ is between 80-90. In another embodiment, n₁ is between 90-100. In another embodiment, n₁ is between 100-110. In another embodiment, n₁ is between 110-120. In another embodiment, n₁ is between 120-130. In another embodiment, n₁ is between 130-140. In another embodiment, n₁ is between 140-150. In another embodiment, n₁ is between 150-160. In another embodiment, n₁ is between 160-170. In another embodiment, n₁ is between 170-180. In another embodiment, n₁ is between 180-190. In another embodiment, n₁ is between 190-200. Each possibility represents a separate embodiment of this invention.

In one embodiment, n₂ of structures (I) and (II) to (VII) is between 0-200. In another embodiment, n₂ is between 0-10. In another embodiment, n₂ is between 10-20. In another embodiment, n₂ is between 20-30. In another embodiment, n₂ is between 30-40. In another embodiment, n₂ is between 40-50. In another embodiment, n₂ is between 50-60. In another embodiment, n₂ is between 60-70. In another embodiment, n₂ is between 70-80. In another embodiment, n₂ is between 80-90. In another embodiment, n₂ is between 90-100. In another embodiment, n₂ is between 100-110. In another embodiment, n₂ is between 110-120. In another embodiment, n₂ is between 120-130. In another embodiment, n₂ is between 130-140. In another embodiment, n₂ is between 140-150. In another embodiment, n₂ is between 150-160. In another embodiment, n₂ is between 160-170. In another embodiment, n₂ is between 170-180. In another embodiment, n₂ is between 180-190. In another embodiment, n₂ is between 190-200. Each possibility represents a separate embodiment of this invention.

In one embodiment, n₃ and n₄ of structure (II) are each independently 0 or 1. Each possibility represents a separate embodiment of this invention.

In one embodiment, m of structures (I) and (II) to (VII) is between 1-200. In another embodiment, m is between 1-10. In another embodiment, m is between 10-20. In another embodiment, m is between 20-30. In another embodiment, m is between 30-40. In another embodiment, m is between 40-50. In another embodiment, m is between 50-60. In another embodiment, m is between 60-70. In another embodiment, m is between 70-80. In another embodiment, m is between 80-90. In another embodiment, m is between 90-100. In another embodiment, m is between 100-110. In another embodiment, m is between 110-120. In another embodiment, m is between 120-130. In another embodiment, m is between 130-140. In another embodiment, m is between 140-150. In another embodiment, m is between 150-160. In another embodiment, m is between 160-170. In another embodiment, m is between 170-180. In another embodiment, m is between 180-190. In another embodiment, m is between 190-200. Each possibility represents a separate embodiment of this invention.

In another embodiment, the anti-microbial active group of this invention may be selected from: (a) a quaternary ammonium group comprising at least one terpenoid moiety; and (b) a pyridinium group. Each possibility represents a separate embodiment of this invention.

In one embodiment, the particles of this invention represented by structures (I)-(VII) comprise an anti-microbial active unit and an inert core, wherein the anti-microbial active unit and the core are linked directly or indirectly.

In some embodiments L₁, L₂ or L₃ is each independently the same or a different linker. In some embodiments, L₁, L₂ and L₃ are connected to each other, in any possible way. In some embodiment, L₃ is nothing and L₁ or L₂ is connected to the core covalently. In another embodiment, L₃ is connected to the core covalently and L₁ or L₂ is connected to L₃. In another embodiment, L₁ is connected to X₁, L₂ and L₃ or core. In another embodiment, a “linker” comprises any possible chemical moiety capable of connecting at least two other chemical moieties which are adjacent to such linker. In another embodiment, the monomeric unit of the anti-microbial active unit comprises a first and/or second linker/s (L₁ or L₂) and an anti-microbial group. In another embodiment, L₁ and/or L₂ are/is the backbone (they are e.g. alkylene, polypeptide or oligosiloxane (—Si(OH)₂—O— or —Si(CH₃)₂—O—) moieties) of the anti-microbial active unit. In some embodiments, the linker comprises a functional group. In another embodiment, the linker comprises two (same or different) functional groups. In another embodiment, the functional group comprises phosphate, phosphonate, siloxane, silane, ether, acetal, hydroxyl, amide, amine, anhydride, ester, ketone, or aromatic ring or rings functionalized with any of the preceding moieties. Each possibility represents a separate embodiment of this invention.

In another embodiment, L₁, L₂, L₃, X₁, X₂, X₃, X₄ or any combination thereof is a C₁ to C₁₈ alkylene, alkenylene, alkynylene or aryl substituted with at least one carboxyl moiety, wherein the carboxyl end is attached to the core. It may be derived from a C₁ to C₁₈ alkylene substituted with at least one carboxyl moiety and having an amino end which is modified to anti-microbial active group [—⁺N(R₁)(R₂)(R₃), —⁺N(R₁′) (R₂′) (R₃′),

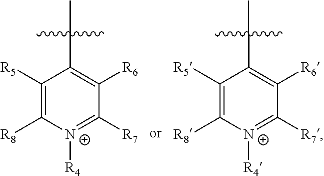

defined in structures (I) and (Ia)]. This linker may be derived from an amino acid of natural or synthetic source having a chain length of between 2 and 18 carbon atoms (polypeptide), or an acyl halide of said amino acid. Non-limiting examples for such amino acids are 18-amino octadecanoic acid and 18-amino stearic acid. In another embodiment, L₁, L₂, L₃, X₁, X₂, X₃, X₄ or any combination thereof is a C₁ to C₁₈ alkylene substituted with at least one amine, amide or pyridinium

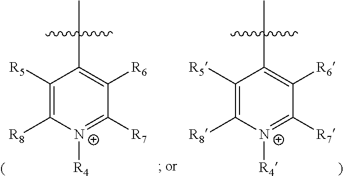

moiety.

In another embodiment, L₁, L₂, L₃, X₁, X₂, X₃, X₄ or any combination thereof is a C₁ to C₁₈ alkylene, alkenylene, alkynylene, arylene or aryl. This linker may be derived from a di-halo alkylene or di-haloarylene, which is functionalized at each end with the core and anti-microbial active group, respectively, by replacement of the halogen moiety to a functional group that binds to the core and replacement of the halogen moiety to obtain —⁺N(R₁)(R₂)(R₃) or —⁺N(R₁′)(R₂′)(R₃′), which are defined in structures (I) to (II).

In another embodiment, L₁, L₂, L₃, X₁, X₂, X₃, X₄ or any combination thereof is an aromatic group derived from non-limiting examples of 4,4-biphenol, dibenzoic acid, dibenzoic halides, dibenzoic sulphonates, terephthalic acid, tetrphthalic halides, and terephthalic sulphonates. This linker is functionalized with the core and anti-microbial active group, respectively, through the functional group thereof (i.e., hydroxyl, carboxy or sulfonate). In another embodiment, this linker is directly attached to the core at one end or indirectly, via a third linker (L₃) and is modified at the other end to anti-microbial active group [—⁺N(R₁)(R₂)(R₃), —⁺N(R₁′)(R₂′)(R₃′),

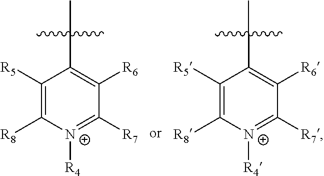

defined in structures (I) and (Ia)].

In another embodiment, L₁, L₂, L₃, X₁, X₂, X₃, X₄ or any combination thereof, is a siloxane or silane group derived and/or selected from non-limiting examples of trialkoxyalkylsilane, trialkoxyarylsilane, trihaloalkylsilane, trihaloarylsilane, 3-aminopropyltriethoxysilane (APTES), (3-glycidyloxypropyl)trimethoxysilane and N-2-aminoethyl-3-aminopropyl trimethoxysilane. In another embodiment, this siloxane or silane group comprises a functional group (e.g. hydroxyl, siloxane, carboxy, amide or sulfonate). In another embodiment, this siloxane or silane group is directly attached to the core at one end directly (without L₃) or indirectly, via a third linker (L₃) and is modified at the other end to anti-microbial active group [—⁺N(R₁)(R₂)(R₃), —⁺N(R₁′) (R₂′) (R₃′),

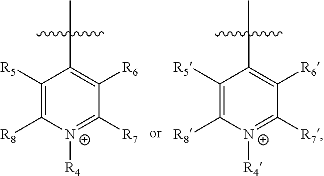

defined in structures (I) and (Ia)].

In another embodiment, L₁, L₂, L₃, X₁, X₂, X₃, X₄ or any combination thereof is a monomeric unit (as described in e.g. FIGS. 1B-1C and formulas Ia and I-VI) within the anti-microbial active unit of this invention and is represented by the structure of formula Ib1 or Ib2:

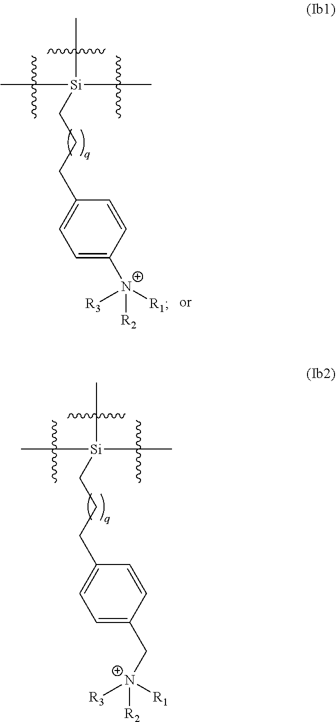

wherein
R₁ and R₂ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R is alkyl, aryl, cycloalkyl, heterocycle or any combination thereof;
q is an integer between 0 and 16; and
wherein said monomeric unit is chemically bound to the surface of an inorganic core directly or via a third linker (L₃).

In another embodiment, L₁, L₂, L₃, X₁, X₂, X₃, X₄ or any combination thereof is a monomeric unit (as described in e.g. FIGS. 1B-1C and formulas Ia and I-VI) within the anti-microbial active unit of this invention and is represented by the structure of formula Ic1 or Ic2:

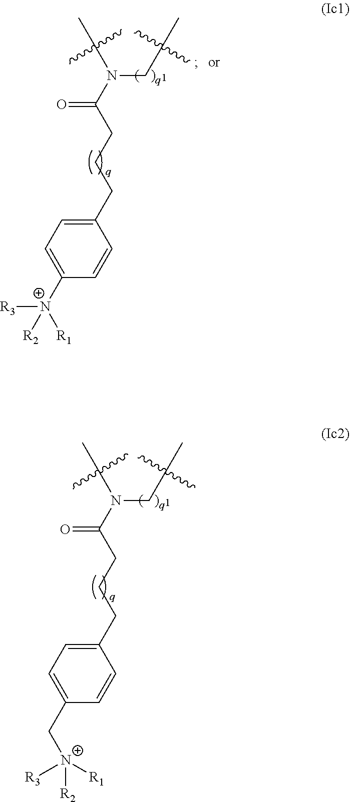

wherein
R₁-R₃ are as described hereinabove;
q and q¹ are independently an integer between 0 and 16; and
wherein said monomeric unit is chemically bound to the surface of an inorganic core directly or via a third linker (L₃).

In another embodiment, a linker molecule which might be used in the processes of preparing the anti-microbial particles of this invention is represented by the structure of formula Id1 or Id2:

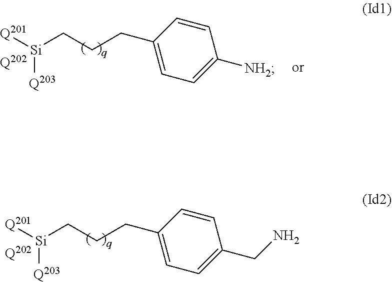

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide;
q is an integer between 0 and 16;
the linker molecule is capable of being chemically bound to the surface of the inorganic core through the silicon atom; and
the anti-microbial active group is introduced by functionalizing the primary amine to obtain an anti-microbial active quaternary ammonium group as described above.

In another embodiment, a linker molecule which might be used in the processes of preparing the anti-microbial particles of this invention is represented by the structure of formula Ie:

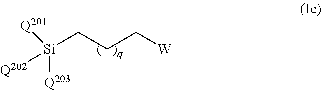

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide;
W is selected from the group consisting of arylene-NH₂, benzylene-NH₂, halide, sulfonate and hydroxyl;
q is an integer between 0 and 16;
said linker is capable of being chemically bound to the surface of said inorganic core through the silicon atom; and the anti-microbial active group is introduced by substituting the group W with an anti-microbial active group, or converting the group W to an anti-microbial active group.

The particles of this invention demonstrate an enhanced anti-microbial activity. Without being bound by any theory or mechanism, it can be postulated that such activity originates from the presence of closely packed anti-microbial groups on a given core's surface, as well as high density of particles packed on the surface of a host material. This density increases as each anti-microbial active unit in the particles of this invention comprise increasing number of anti-microbial active groups and it yields a high local concentration of active functional groups, which results in high effective concentration of the anti-microbial active groups and enables the use of a relatively small amount of particles to achieve effective bacterial annihilation. The close packing of the anti-microbial groups is due to, inter alia, numerous anti-microbial active units protruding from each particle surface. Accordingly, the anti-microbial groups cover large fraction of the particle's available surface area (width dimension covering the surface). The surface density of the anti-microbial group results in high effective concentration promoting anti-microbial inhibitory effect. According to the principles of this invention, high surface density dictates high anti-microbial efficiency.

The term “nanoparticle” as used herein refers to a particle having a diameter of less than about 1,000 nm. The term “microparticle” as used herein refers to a particle having a diameter of about 1,000 nm or larger.

The anti-microbial particles of this invention are characterized by having a diameter between about 5 to about 100,000 nm, and thus encompass both nanoparticulate and microparticulate compositions. Preferred are particles between about 10 to about 50,000 nm. In other embodiments, the particles are more than 1,000 nm in diameter. In other embodiments, the particles are more than 10,000 nm in diameter. In other embodiment, the particles are between 1,000 and 50,000 nm in diameter. In other embodiment, the particles are between 5 and 250 nm in diameter. In other embodiment, the particles are between 5 and 500 nm in diameter. In another embodiment, the particles are between 5 and 1000 nm in diameter. It is apparent to a person of skill in the art that other particles size ranges are applicable and are encompassed within the scope of this invention.

Anti-Microbial Active Groups Comprising Terpenoid Groups

In one embodiment, the anti-microbial active group of this invention contains at least one terpenoid group. In one embodiment, R₃, R₅-R₈, R₃′ and/or R₅′-R₈′ of the anti-microbial active groups [—⁺N(R₁)(R₂)(R₃), —⁺N(R₁′)(R₂′)(R₃′),

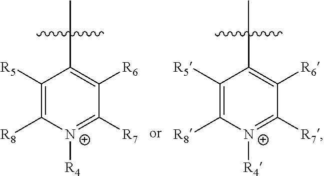

defined in structures (I) and (Ia)] are the terpenoid moieties. In another embodiment, when the anti-microbial active group of this invention contains at least one terpenoid group and/or R₃, R₅—R₈, R₃′ and/or R₅′-R₈′ of the anti-microbial active groups as defined hereinabove are terpenoid moieties—the core is a polyhedral oligomeric silsesquioxane (POSS).

The term “terpenoid”, also known as “isoprenoid” refers to a large class of naturally occurring compounds that are derived from five-carbon isoprene units. A “terpenoid moiety” is derived from a terpenoid.

In some embodiments, the terpenoid moiety is a “terpenoidyl”, i.e. directly bonded to one group (e.g. cinnamyl:

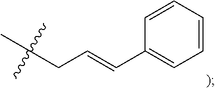

or a “terpenoidylene”, i.e. directly bonded to two groups (e.g. cinnamylene, e.g.

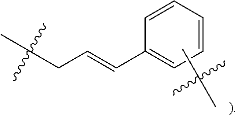

In one embodiment, the terpenoid moiety is directly bonded to more than two groups. In one embodiment, the terpenoid moiety is a cinammyl or cinnamylene group derived from cinnamaldehyde, cinnamic acid, curcumin, viscidone or cinnamyl alcohol. In another embodiment, the terpenoid moiety is a bornyl or a bornylene group derived from camphor, bornyl halide or bornyl alcohol. In another embodiment, the terpenoid moiety is derived from citral. In another embodiment, the terpenoid moiety is derived from perilaldehyde. Each possibility represents a separate embodiment of this invention.

Cinnamaldehyde is a natural aldehyde extracted from the genus Cinnamomum. It is known for its low toxicity and its effectiveness against various bacteria and fungi.

Camphor is found in the wood of the camphor laurel (Cinnamomum camphora), and also of the kapur tree. It also occurs in some other related trees in the laurel family, for example Ocotea usambarensis, as well as other natural sources. Camphor can also be synthetically produced from oil of turpentine. Camphor can be found as an R or S enantiomer, a mixture of enantiomers and a racemic mixture. Each possibility represents a separate embodiment of this invention.

Citral, or 3,7-dimethyl-2,6-octadienal or lemonal, is a mixture of two diastereomeric terpenoids. The two compounds are double bond isomers. The E-isomer is known as geranial or citral A. The Z-isomer is known as neral or citral B. Citral is known to have anti-microbial activity.

Perillaldehyde, also known as perilla aldehyde, is a natural terpenoid found most in the annual herb perilla, as well as in a wide variety of other plants and essential oils.

Other examples of terpenoids include, but are not limited to: curcuminoids found in turmeric and mustard seed, citronellal found in Cymbopogon (lemon grass) and carvacrol, found in Origanurn vulgare (oregano), thyme, pepperwort, wild bergamot and Lippia graveolens. Each possibility represents a separate embodiment of this invention.

In accordance with the above embodiment, the anti-microbial active terpenoid moieties are selected from the group consisting of:

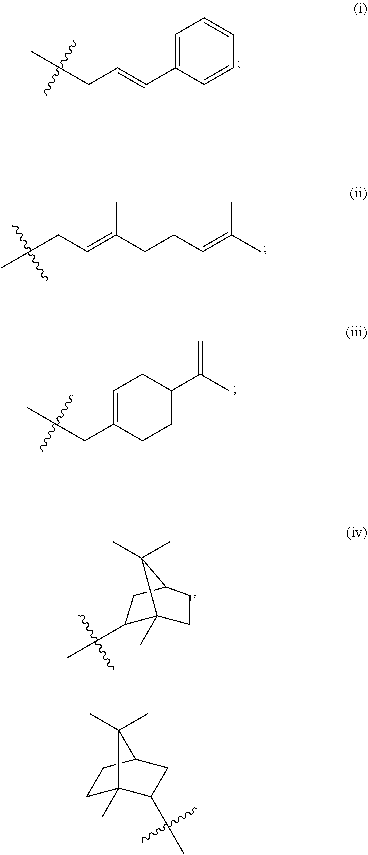 [SMILES: *C/C=C/c1ccccc1 ; *C/C=C(\C)CCC=C(C)C ; *CC1=CCC(C(=C)C)CC1 ; *C1CC2CCC1(C)C2(C)C ; *C1CC2CCC1(C)C2(C)C]

or any combination thereof;

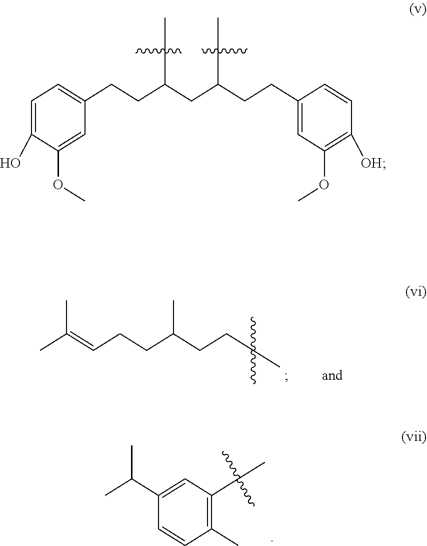 [SMILES: *C(CCc1ccc(O)c(OC)c1)CC(*)CCc1ccc(O)c(OC)c1 ; *CCC(C)CCC=C(C)C ; *c1cc(C(C)C)ccc1C]

Each possibility represents a separate embodiment of this invention.

Non-limiting examples of anti-microbial active quaternary ammonium groups in accordance with the principles of this invention are:

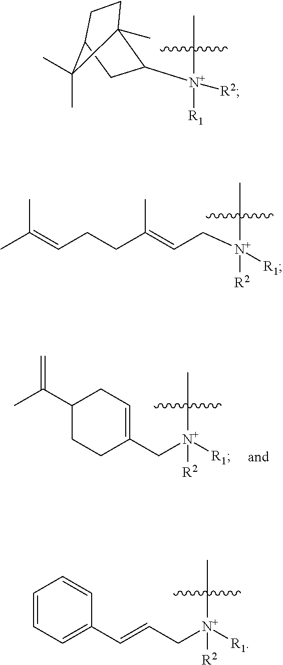

wherein R₁ and R₂ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof; and
R is alkyl, aryl, cycloalkyl, heterocycle or any combination thereof.

The anti-microbial active group of this invention may be in the form of a quaternary ammonium or pyridinium salt, as described hereinabove. Since an such groups are positively charged, their charge is balanced with an anion. Non-limiting examples of anions include: a halide, e.g. fluoride, chloride, bromide or iodide and fluoride, bicarbonate, nitrate, phosphate, acetate, fumarate, succinate, mesylate, triflate, tosylate, tetrafluoroborate, hexafluorophosphate and sulfate. Each possibility represents a separate embodiment of this invention.

Anti-microbial active groups comprising one long alkyl group.

In one embodiment, the anti-microbial active group of this invention contains one alkyl group which have from 4 to 24 carbon atoms as R₅-R₈, and/or R₅′-R₈′ of the anti-microbial active groups

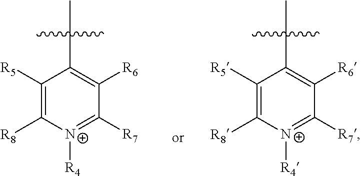

defined in structures (I) and (Ia)].

In another embodiment, the alkyl group has 4-6, 4-8, 4-10, 4-12, 4-14, 4-16, 4-18, 4-20, 4-22, 8-12, 12-16, 16-24, 18-24, 10-24, 10-20 or 10-18 carbon atoms. Each possibility represents a separate embodiment of this invention.

The term “quaternary ammonium group” refers to a group of atoms consisting of a nitrogen atom with four substituents (different than hydrogen) attached thereto. In another embodiment, a “quaternary ammonium group” refers to a group of atoms consisting of a nitrogen atom with four groups wherein each of the group is attached to the nitrogen through a carbon atom. The term “long alkyl group” or chain refers to such an alkyl group or chain which is substituted on the nitrogen atom of the quaternary ammonium group or found as sub stituent to the pyridinum and which has between 4 and 24 carbon atoms. In some currently preferred embodiments, the alkyl group is an alkyl group having 4 to 18 carbon atoms. In some currently preferred embodiments, the alkyl group is an alkyl group having 4 to 10 carbon atoms. In some currently preferred embodiments, the alkyl group is an alkyl group having 12 to 16 carbon atoms. In some currently preferred embodiments, the alkyl group is an alkyl group having 16 to 24 carbon atoms. In some currently preferred embodiments, the alkyl group is an alkyl group having 18 to 24 carbon atoms. In some currently preferred embodiments, the alkyl group is an alkyl group having 10 to 24 carbon atoms. In some currently preferred embodiments, the alkyl group is an alkyl group having 4 to 12 carbon atoms. In some currently preferred embodiments, the alkyl group is an alkyl group having 4 to 8 carbon atoms. In some currently preferred embodiments, the alkyl group is an alkyl group having 4 to 10 carbon atoms. In other currently preferred embodiments, the alkyl group is an alkyl group having 6, 7, or 8 carbon atoms, with each possibility representing a separate embodiment of this invention.

Organic polymeric Cores

In some embodiments, the core of the anti-microbial particles is an organic polymeric core. In one embodiment, the organic core comprises at least one aliphatic polymer. An “aliphatic polymer” as used within the scope of this invention refers to a polymer made of aliphatic monomers that may be substituted with various side groups, including (but not restricted to) aromatic side groups. Aliphatic polymers that may be included in particles according to this invention comprise nitrogen atoms (as well as other heteroatoms) as part of the polymeric backbone. In one embodiment, the core of the particles is an organic polymeric core including amines which can be substituted with R₁, R₂, R₃, R₁′, R₂′ and/or R₃′ as defined for structure I; or including an imine which is chemically modified to amine and then substituted with R₁, R₂, R₃, R₁′, R₂′ and/or R₃′ as defined for structure I. Non-limiting examples of aliphatic polymers are polystyrene (PS), crosslinked PS, chlorinated crosslinked PS, polyvinylchloride (PVC), polyethylene imine (PEI), polyvinyl amine (PVA), poly(allyl amine) (PAA), poly(aminoethyl acrylate), polypeptides with pending alkyl-amino groups, and chitosan. Each possibility represents a separate embodiment of this invention. In one currently preferred embodiment, the polymer is polyethylene imine (PEI).

In another embodiment, the organic core comprises at least one aromatic polymer selected from the following group: polystyrene, aminomethylated styrene polymers, aromatic polyesters, preferably polyethylene terephthalate, and polyvinyl pyridine.

The polymeric core may be linked to anti-microbial active part directly (i.e. in structures (I)-(VII): L₃ is a bond) or via a linker. Each possibility represents a separate embodiment of this invention.

In one embodiment, the organic polymeric core includes a combination of two or more different organic polymers. In another embodiment, the organic polymeric core includes a copolymer.

In some embodiments, anti-microbial active unit is linked to the organic polymeric core directly (L₃ is a bond) or via a linker (L₃). In these embodiments, the linker may be selected from:

(a) a C₁ to C₁₈ alkylene substituted with at least one carboxyl moiety. This linker may be derived from an alkylene substituted with at least one carboxyl moiety and at least one amino moiety, wherein the carboxyl end is attached to the core and the amino end is modified to anti-microbial active group [—⁺N(R₁)(R₂)(R₃), —⁺N(R₁′)(R₂′)(R₃′),

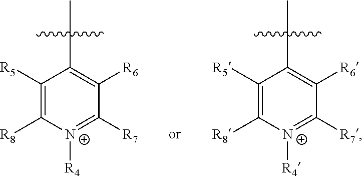

defined in structures (I) and (Ia)]. This linker may be derived from an amino acid of natural or synthetic source having a chain length of between 2 and 18 carbon atoms, or an acyl halide of said amino acid. Non-limiting examples for such amino acids are 18-amino octadecanoic acid and 18-amino stearic acid;
(b) a C1 to C₁₈ alkylene. This linker may be derived from a di-halo alkylene, which is functionalized at each end with the core and anti-microbial active group, respectively, by replacement of the halogen moiety to a functional group that will bind to the core and replacement of the halogen moiety to obtain [—+N(R₁)(R₂)(R₃) or —⁺N(R₁′) (R₂′)(R₃′), defined in structures (I) and (Ia)]; and (c) aromatic molecules derived from 4,4-biphenol, dibenzoic acid, dibenzoic halides, dibenzoic sulphonates, terephthalic acid, tetrphthalic halides, and terephthalic sulphonates. This linker is functionalized with the core and anti-microbial active group, respectively, through the functional group thereof (i.e., hydroxyl, carboxy or sulfonate). In another embodiment, this linker is attached to the core at one end and is modified at the other end to anti-microbial active group [—⁺N(R₁)(R₂)(R₃), —⁺N(R₁′)(R₂′)(R₃′),

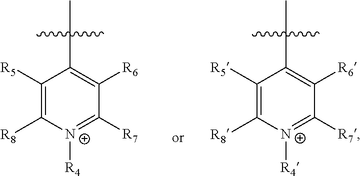

defined in structures (I) and (Ia)]. In another embodiment, the linker comprises alkyl, alkenyl, alkyl phosphate, alkyl siloxanes, carboxylate, epoxy, acylhalides and anhydrides, or combination thereof, wherein the functional group is attached to the core. Each possibility represents a separate embodiment of this invention.

Various polymeric chains may provide a range of properties that themselves may be an accumulation of the various polymer properties, and may even provide unexpected synergistic properties. Examples of such mixed polyamine particles include: crosslinking of aliphatic and aromatic polyamines such as polyethyleneimine and poly(4-vinyl pyridine) via a dihaloalkane; mixture of linear short chain and branched high molecular weight polyethyleneimines; interpenetrating compositions of polyamine within a polyamine scaffold such as polyethyleneimine embedded within crosslinked polyvinyl pyridine particles, or even interpenetrating a polyamine into a low density non-amine scaffold such as polystyrene particles. In other words, the use of polyamine combinations for the purpose of forming particles, either by chemical crosslinking or physical crosslinking (interpenetrating networks) may afford structures of varying properties (such as being able to better kill one bacteria vs. another type of bacteria). Such properties may be additive or synergistic in nature.

In one specific embodiment, the organic polymeric core is cross-linked with a cross-linking agent. The preferred degree of cross-linking is from 1% to 20%, when crosslinking of from about 2% to about 5% is preferable. The crosslinking may prevent unfolding of the polymer and separation of the various polymeric chains that form the particle.

Crosslinking, as may be known to a person skilled in the art of organic synthesis and polymer science, may be affected by various agents and reactions that are per se known in the art. For example, crosslinking may be affected by alkylating the polymer chains with dihaloalkane such as dibromoethane, dibromocyclohexane, or bis-bromomethylbenzene. Alternatively, crosslinking by reductive amination may be used. In this method a polyamine with primary amines is reacted with a diketone or with an alkane dialdehyde to form an imine crosslinker which is then further hydrogenated to the corresponding amine. This amine is further reacted to form an antimicrobial effective quaternary ammonium group. In such a method, instead of dihaloalkanes or dialdehydes, tri or polyhaloalkanes or polyaldehydes or polyketones are used.

The preferred polymers useful for making the polymeric core according to this invention are those having chains made of 30 monomer units, preferably 100 monomer units that may be crosslinked using less than 10% of crosslinking agent. The longer the polymers are, the fewer crosslinking bonds are needed to afford an insoluble core. Branched polymers are preferred for crosslinking as small amount of crosslinking is required to form insoluble core.

In some embodiments, at least about 10% of the amine groups in the organic polymeric core are the anti-microbial active tertiary amine/ammonium or quaternary ammonium groups or salts thereof, as described herein.

In one embodiment, the anti-microbial particles according to this invention have functional groups that are capable of reacting with a host polymer or with monomers thereof. Such functional groups are designed to allow the particles to be bound chemically to a hosting material.

Inorganic Cores

In some embodiments, the core of the anti-microbial particles of this invention is an inorganic core comprising one or more inorganic materials. Inorganic cores have a few advantages over organic polymeric cores: 1) higher stability at elevated temperature; 2) higher chemical stability towards various solvent and reagents; 3) improved mechanical strength; 4) better handling qualities in composites due to their amphipathic nature; and 5) lower cost.

An additional advantage of inorganic cores relates to the insertion of the functionalized particles into a polymeric material within a polymeric matrix (host). In contrast to organic cores which are produced by radical polymerization (e.g. acrylate resins), inorganic cores do not interfere with the polymerization process and hence do not jeopardize the mechanical properties of the finalized substrate, as opposed to organic polymeric cores which tend to interfere with the polymerization reaction.

In one embodiment, the inorganic core comprises silica, glass, glass powder, metal, metal oxide, ceramic material or a zeolite. Each possibility represents a separate embodiment of this invention.

In one embodiment, the core of the particles of this invention comprises silica (SiO₂). The silica may be in any form known in the art, non-limiting examples of which include polyhedral oligomeric silsesquioxane (POSS), amorphous silica, dense silica, aerogel silica, porous silica, mesoporous silica and fumed silica.

The surface density of active groups onto particle surface have proportional impact on its anti-microbial activity. This is applicable both to organic and inorganic particles in same manner. In another embodiment, the core of the particles of this invention comprises glasses or ceramics of silicate (SiO₄⁻⁴). Non-limiting examples of silicates include aluminosilicate, borosilicate, barium silicate, barium borosilicate and strontium borosilicate.

In another embodiment, the core of the particles of this invention comprises surface activated metals selected from the group of: silver, gold, platinum, palladium, copper, zinc and iron.

In another embodiment, the core of the particles of this invention comprises metal oxides selected from the group of: zirconium dioxide, titanium dioxide, vanadium dioxide, zinc oxide, copper oxide and magnetite.

The inorganic core typically has a solid uniform morphology with low porosity or a porous morphology having pore size diameter of between about 1 to about 30 nm.

In another embodiment, the core of the particles of this invention comprises natural or artificial Zeolites.

In one embodiment, non-limiting examples of ceramic materials include: oxides (e.g. zinc oxide, boron oxide, zirconium oxide), carbides (e.g. silicon carbide, titanium carbide), nitrides (e.g. titanium nitride, boron nitride) and borides (e.g. magnesium diboride)

In one embodiment, the core may be attached to the anti-microbial unit directly (i.e. in structures (I)-(VII): L₃ is a bond), or via a linker (L₃). Preferably a silica (SiO₂) based inorganic core may be attached to the anti-microbial part through a linker (L₃), while glasses or ceramicas of silicate (SiO₄⁻⁴), metals or metal oxides may be attached to anti-microbial unit directly (i.e. in structures (I)-(VII): L₃ is a bond).

In some embodiments, the inorganic core is directly (i.e. in structures (I)-(VII): L₃ is a bond) attached to the anti-microbial unit. In other embodiments, the inorganic core is attached to the anti-microbial unit through a linker. In some embodiments, the linker is selected from the following groups: a C1 to C18 alkylene; a C1 to C18 alkylene substituted with at least one silane or alkoxysliane moiety; a C1 to C18 alkylene substituted with at least one phosphate moiety; a C1 to C18 alkylene substituted with at least one anhydride moiety; a C1 to C18 alkylene substituted with at least one carboxylate moiety; and a C1 to C18 alkylene substituted with at least one glycidyl moiety. Each possibility represents a separate embodiment of this invention.

The inorganic core of the particle as described above may generally be in a form selected from a sphere, amorphous polygonal, shallow flake-like and a rod. In some representative embodiments, the inorganic core is spherical and has a diameter between about 5 to about 100,000 nm. In some representative embodiments, the inorganic core is spherical and has a diameter between about 1000-100,000 nm. In some representative embodiments, the inorganic core is spherical and has a diameter between about 100-1000 nm with pore diameter of about 1 to about 100 nm. In another embodiment, the inorganic spherical core has a pore diameter of about 1 to about 50 nm. In another embodiment, the inorganic spherical core has a pore diameter of about 1 to about 30 nm. In another embodiment, the inorganic particle is in a form of a rod, having a diameter of between about 5 to about 1,000 nm and length between about 10 to about 1,000,000 nm. In another embodiment, a length of between 50 to 100,000 nm. In another embodiment, a length of between 100 to 250,000 nm. In another embodiment, a length of between 200 to 500,000 and a pore diameter of about 1 to about 50 nm. Each possibility represents a separate embodiment of this invention.

Preparation of anti-microbial particles, comprising one monomeric unit per one anti-microbial active part

The particles of this invention may be prepared in accordance to a variety of processes, depending on the nature of the core, the anti-microbial active group, and the presence or absence of linkers. Some non-limiting examples of preparation methods are provided below.

In one embodiment, this invention provides processes for preparing anti-microbial particles, wherein the particles comprise one monomeric unit per one anti-microbial active unit. In the following, such processes will be presented in detail.

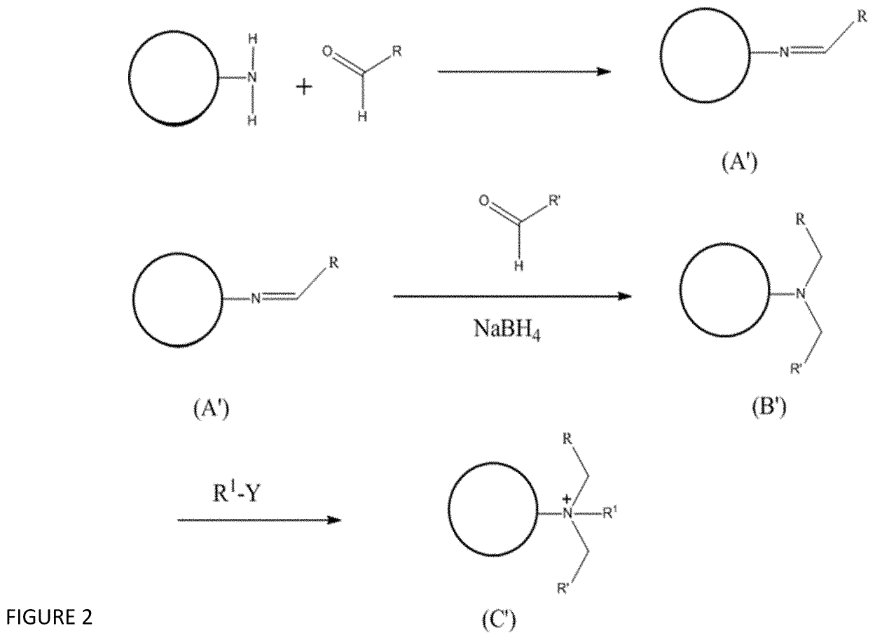

A representative method for preparing particles according to this invention wherein the anti-microbial active group is a quaternary ammonium group is represented in FIG. 2. In accordance with FIG. 2, a core as defined herein is functionalized with a primary amine. The primary amine reacts with an aldehyde to yield initially an imine (Schiff base) intermediate of formula (A′), which is then reacted with a second aldehyde under reductive amination conditions to yield a tertiary amine of formula (B′). R and R′ are each independently methyl, CF₃, perhaloalkyl, aryl, —C(═O)OR, —C(═O)OC(═O)R, —C(═S)OR, —C(═O)SR, —C(═O)—R, —C(═S)—R, 1-alkenyl or 1-alkynyl, where R is alkyl, aryl, cycloalkyl, heterocycle or any combination thereof. Conversion of the tertiary amine to the quaternary ammonium group involves reaction of the tertiary amine with a group R¹—Y wherein R¹ is a methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, and Y is a leaving group such as halogen or sulfonate, where R is alkyl, aryl, cycloalkyl, heterocycle or any combination thereof.

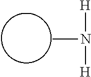

It is understood that the group may represents any one or more of the following:
1. An organic core directly bound to NH₂.
2. An organic core bound to NH₂ through a linker as described herein.
3. An inorganic core directly bound to NH₂.
4. An inorganic core bound to NH₂ through a linker as described herein.

The exemplified reaction (FIG. 2) may be a “one pot synthesis”, or it may include two sequential reactions with isolation of an intermediate formed in the first step. The first step is the formation of intermediate (A′), which is an imine (Schiff base), by reacting an amine functionalized core with a RCHO in the presence of a reducing agent. The imine functionalized core can be isolated at this stage if desired. Alternatively, further reacting intermediate (A′) with R′CHO in the presence of a reducing agent yields a tertiary amine comprising R and R′ moieties (B′). In order to obtain the quaternary ammonium, additional alkylation step is performed as described in FIG. 2.

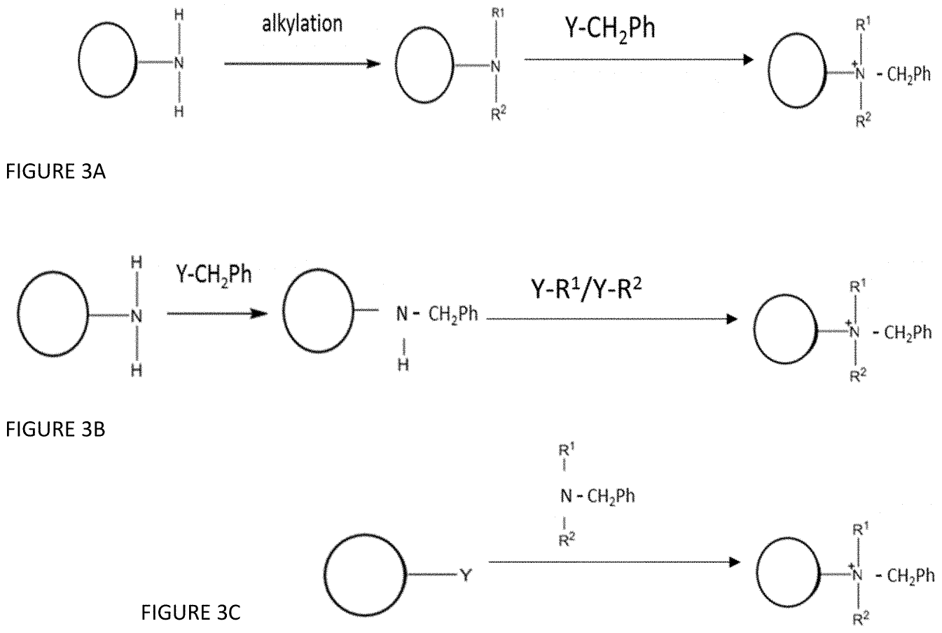

A representative method for preparing particles according to this invention wherein the anti-microbial active group is a quaternary ammonium group containing one benzyl group is presented in FIGS. 3A-C. The method includes three pathways to prepare quaternary ammonium salts (QAS) functionalized particle. FIG. 3A) by reaction with R₁—Y/R₂—Y to achieve tertiary amine, followed by benzylation reaction; FIG. 3B) by a similar pathway as in FIG. 3A), done in the reversed order; and FIG. 3C): by reacting a linker functionalized with a leaving group (e.g., Cl or other halogen) with tertiary amine. R₁ and R₂ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof. Y represents any leaving group, for example Cl, Br or I, or a sulfonate (e.g., mesyl, tosyl).

It is understood that that the group

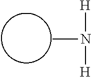

has any one of the meanings as described above for FIG. 2.

It is understood that that the group

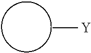

may represents any one or more of the following:
1. An organic core directly bound to Y.
2. An organic core bound to Y through a linker as described herein.
3. An inorganic core directly bound to Y.
4. An inorganic core bound to Y through a linker as described herein.

Core functionalization can occur by a solid support method, or a solution method.

Solid support as method of preparation of anti-microbial particles comprising one monomeric unit per one anti-microbial active part

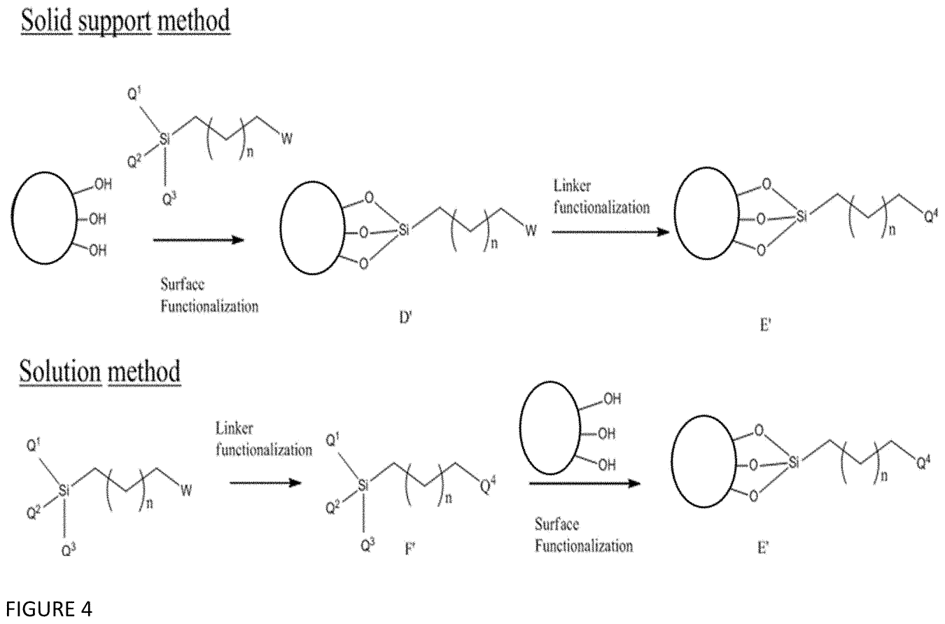

Preparation of functionalized particles is conducted in two general steps. First, the linker molecule is allowed to condense onto particles surface (surface functionalization) via hydrolysis of leaving groups to give an intermediate of formula D′ (FIG. 4, D′). Second, functional sites of the linker molecule undergo further functionalization (linker functionalization) as mentioned in any ones of (FIGS. 2-3) to give a functionalized particle of formula (E′).

Solution method as method of preparation of anti-microbial particles comprising one monomeric unit per one anti-microbial active part

In this method, the linker molecule is first functionalized with antimicrobial active group to give an intermediate of formula (FIG. 4, F′). In the second stage intermediate (F′) is allowed to settle onto particle's solid surface (surface functionalization) to give a functionalized particle of formula (FIG. 4, E′).

Preparation of anti-microbial particles, comprising more than one monomeric unit per one anti-microbial active unit

In one embodiment, this invention provides processes for preparing particles of the composites of this invention, wherein the particles comprise more than one monomeric unit per one anti-microbial active unit. In the following, such processes will be presented in detail.

Solid support as method of preparation of anti-microbial particles comprising more than one monomeric unit per one anti-microbial active unit

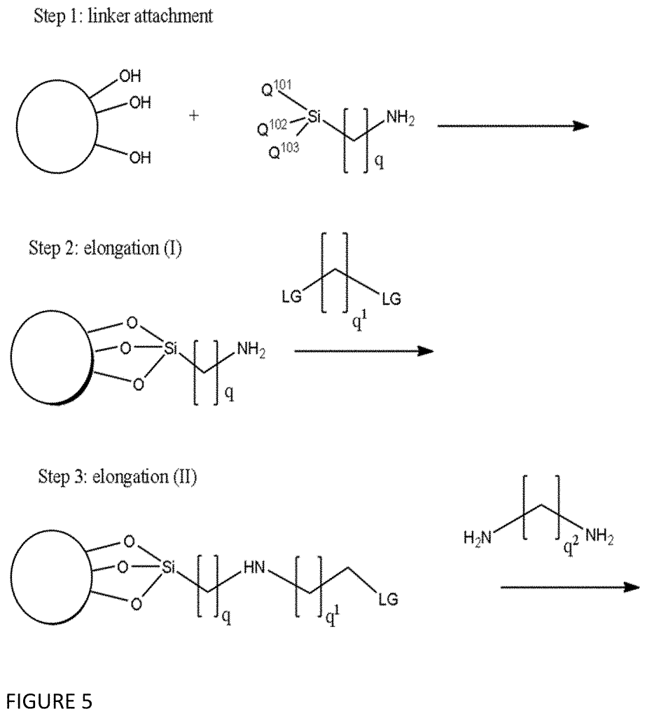

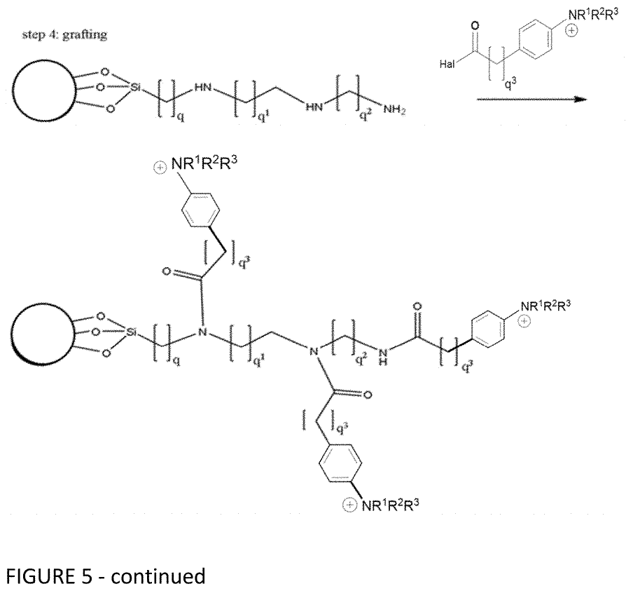

The solid support method comprises a few stages. First, the linker molecule (dilute solutions of a few percent) is allowed to condense onto particles surface (surface functionalization) via (acid catalyzed) hydrolysis of leaving groups, resulting in the attachment of the linker to the core (FIG. 5, step 1). Second, the attached linker is elongated. In another embodiment, this stage is achieved synthetically via one step or more. In another embodiment, elongation is achieved by consecutive addition of difunctionalized alkane and diaminoalkane, wherein amines (of attached linker and diaminoalkane) attack electrophilic centers of the difunctionalized alkane (FIG. 5, steps 2 and 3). In another embodiment, such consecutive addition is optionally repeated for 1-10 times. Finally, the anti-microbial active group (usually attached to an arylene) is grafted to resulting attached and elongated linker. In another embodiment, grafting is accomplished when amines on the attached and elongated linker attack acyl halide moiety of the molecule of the anti-microbial active group which is grafted (FIG. 5, step 4).

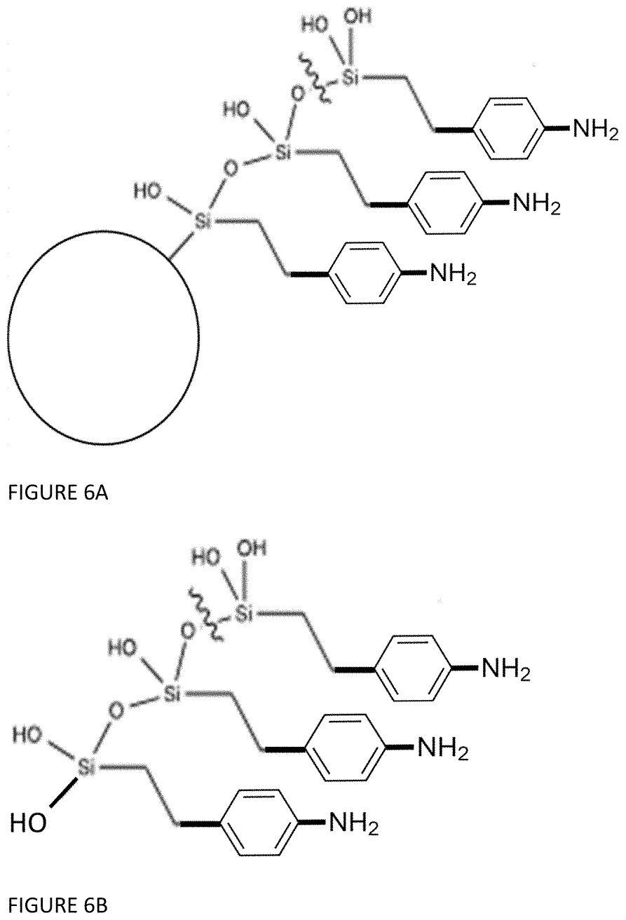

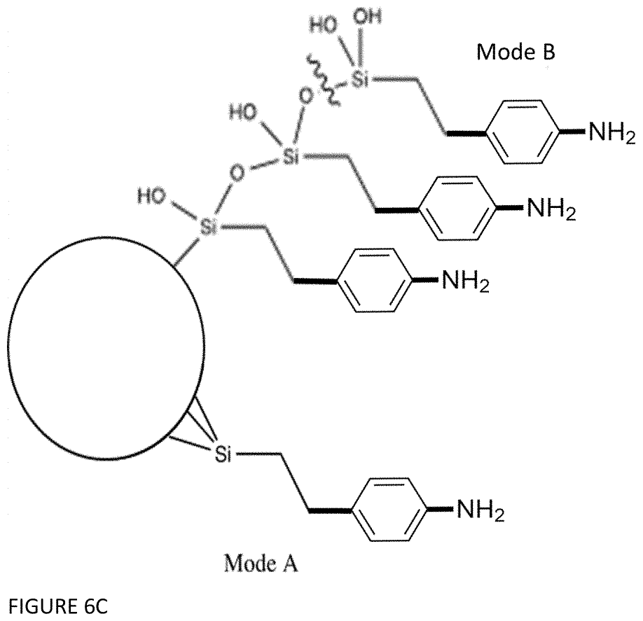

In another embodiment, the same trialkoxysilane linker molecule is used initially, however in a higher concentration (≥10% by wt) and it initially self-polymerizes (FIG. 6A) under basic catalysis. Functionalization of the solid supported linker progresses similarly as in the procedures described hereinabove for particles that comprise one monomeric unit per one anti-microbial active unit (FIGS. 2-4).

Solution method as method of preparation of anti-microbial particles comprising more than one monomeric unit per one anti-microbial active unit

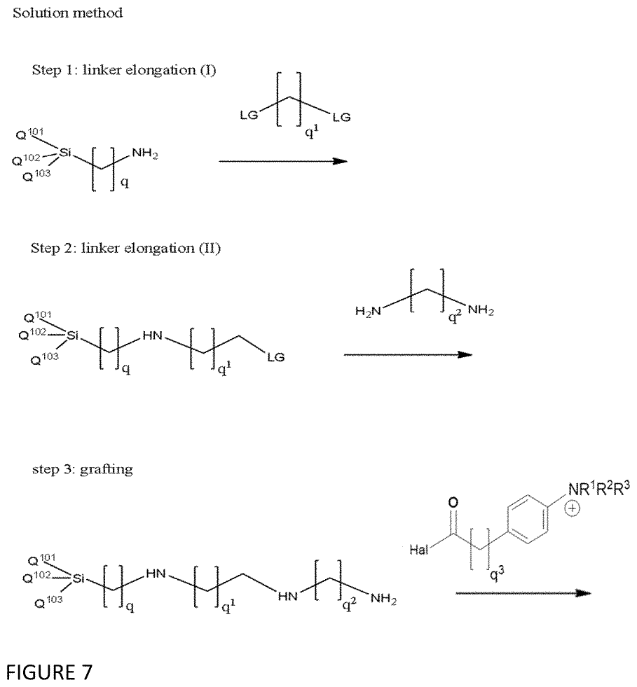

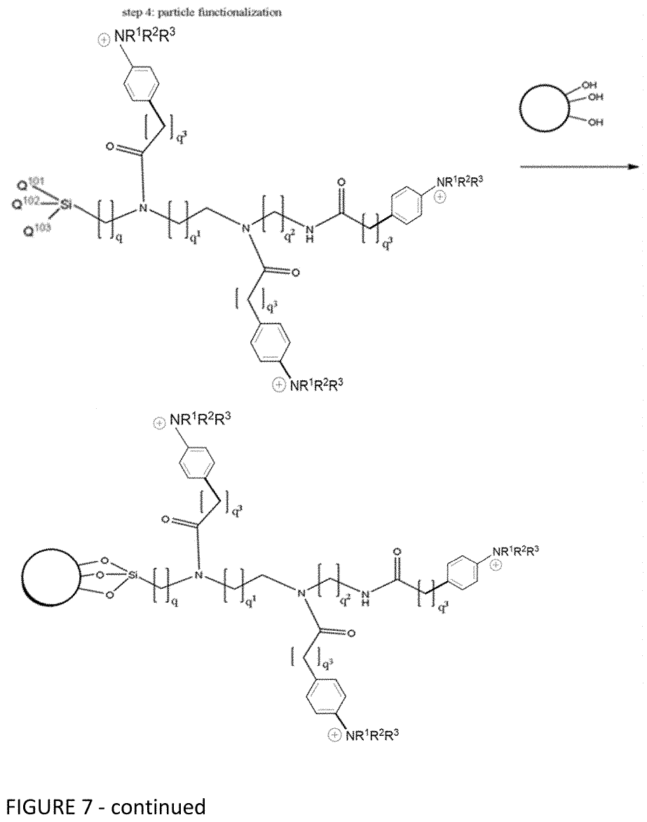

The solution method comprises a few stages. The first step involves elongation of the linker molecule. In another embodiment, this step is achieved synthetically via one step or more. In another embodiment, elongation is achieved by consecutive addition of difunctionalized alkane and diaminoalkane wherein amines (of linker and diaminoalkane) attack electrophilic centers of the difunctionalized alkane (FIG. 7, steps 1 and 2). In another embodiment, such consecutive addition is optionally repeated for 1-10 times. In the second stage, the anti-microbial active group (usually attached to an arylene) is grafted to resulting elongated linker. In another embodiment, grafting is accomplished when amines on the elongated linker attack acyl halide moiety of the molecule of the anti-microbial active group which is grafted (FIG. 7, step 3). Finally, the elongated, anti-microbial active linker is attached to the core via functionalization thereof. In this step, the linker molecule (dilute solutions of a few percent) is allowed to condense onto particles surface (surface functionalization) via (acid catalyzed) hydrolysis of leaving groups, resulting in the attachment of the linker to the core (FIG. 7, step 4).

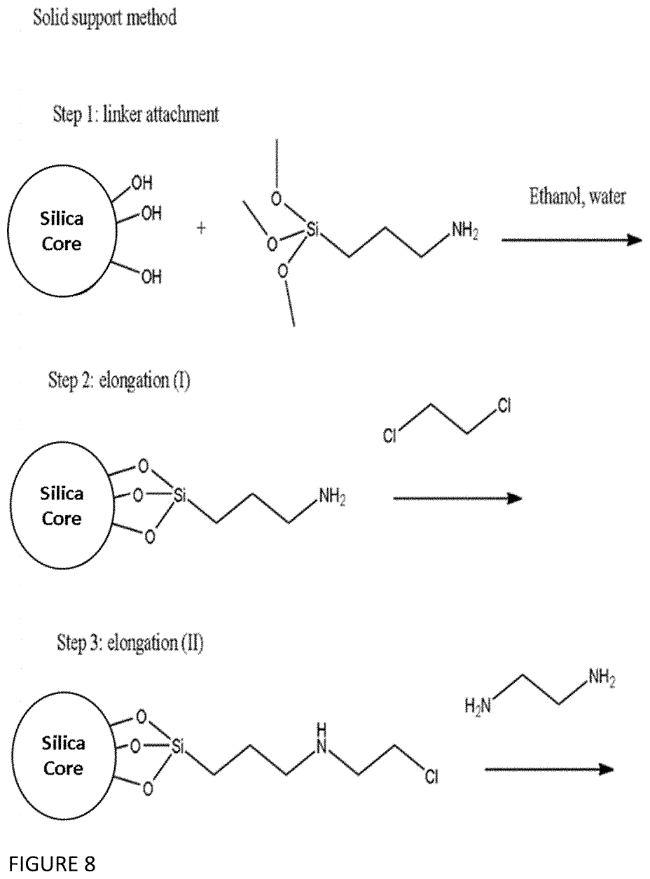

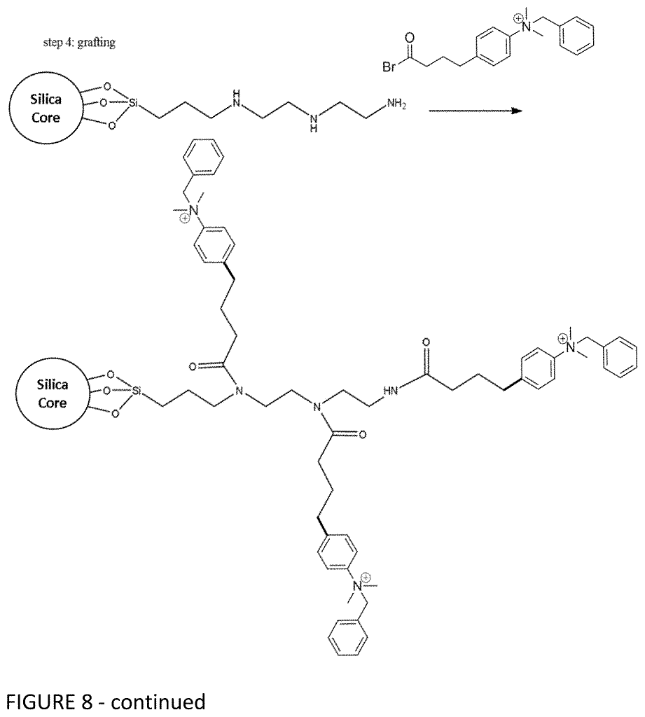

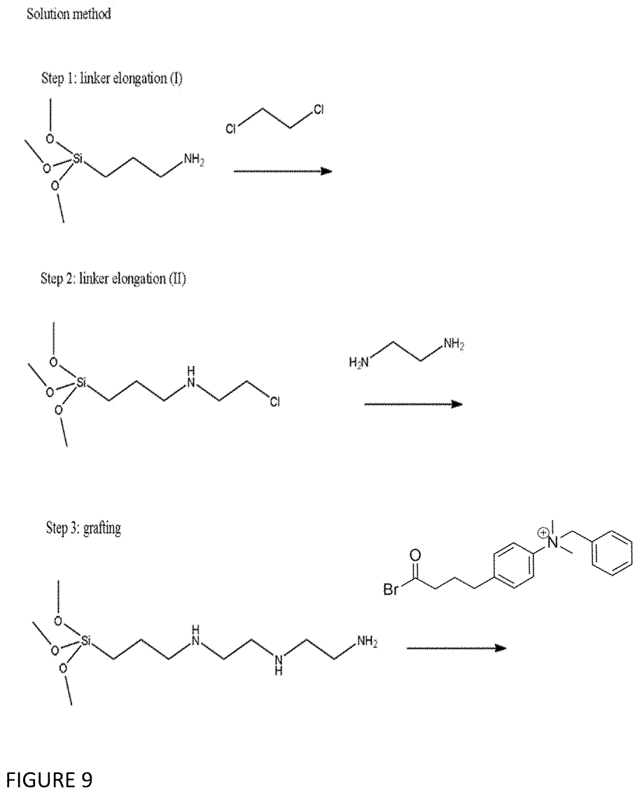

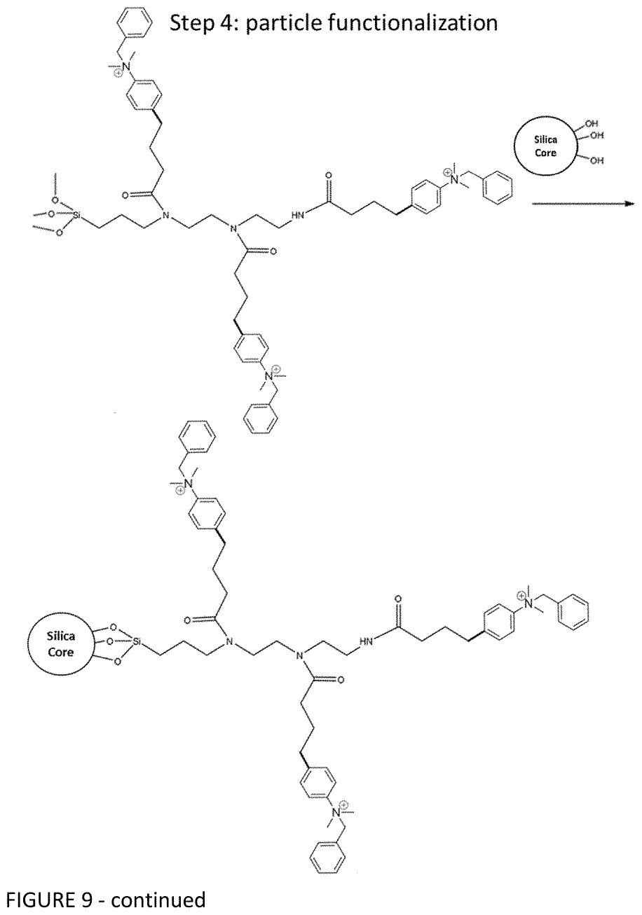

This process is exemplified in FIGS. 8-9—for silica functionalized with dimethybenzylammonium, but is applicable to other hydroxyl-terminated cores and anti-microbial active groups.

In another embodiment, the same trialkoxysilane linker molecule is used initially, however in a higher concentration (10% by weight) and it initially self-polymerizes (FIG. 6B) under basic catalysis. Functionalization of the linker progresses similarly as in the procedures described hereinabove for particles that comprise one monomeric unit per one anti-microbial active part (FIGS. 2-4).

Preparation of Core Particles

In some embodiments, the particles of the composites of this invention which comprise one or more monomeric units per one anti-microbial active part, comprise cores which are prepared according to the following.

Porous silica materials can be prepared by reaction of SiCl₄ with alcohol or water, followed by drying using centrifugation and/or heating utilizing airflow or under vacuum conditions. Dense fumed silica particles (pyrogenic) were prepared by pyrolysis of SiC1₄.

An alternative preparation method of silica core material can be carried by the hydrolysis of tetraethylorthosilicate (TEOS) or tetramethyl orthosilicate (TMS) in the presence of alcohol or water solution and under basic (Stober) or acidic catalytic conditions.

Mesoporous silica particles can be prepared by hydrolysis of TEOS or TMS at low temperatures, preferably in a temperature not exceeding 60° C., followed by dehydration by centrifugation and/or evaporation under airflow or vacuum conditions.

Dense particles can be prepared utilizing intense heating in a process called calcination. Typically, such process takes place at high temperatures at about 250° C.

Composition comprising the particles of this invention

In some embodiments, the composition of this invention comprises the anti-microbial particles of this invention and a polymeric material comprising organic polymers, inorganic polymers or any combination thereof. In some embodiment, the particles as described herein are dispersed in the polymeric material. In another embodiment, the particles are homogeneously dispersed within the polymeric material. In another embodiment, the particles are found in the surface of the polymeric materials. In another embodiment, the particles coat the polymeric materials. In another embodiment, the particles interact weakly or physically (mechanically) with the polymeric material. In another embodiment, the anti-microbial particles are mechanically embedded within the polymeric material. In another embodiment, these particles are three dimensionally “locked” between the polymer chains, preventing them from migrating out from the complex network. The strong hydrophobic nature of these particles also plays a role in preventing the particles from moving into the hydrophilic surrounds such as in the case of physiological, dental, orthopedic or other medical applications. In another embodiment, the polymeric material is inert to the particles and does not react with them. In one embodiment, the particles comprise functional groups, capable of reacting with moieties of the polymeric material. In another embodiment, the particles interact chemically with the polymeric material. In another embodiment, the particles are a mixture of different particles.

In some embodiments, the composition of this invention comprises the anti-microbial particles of this invention and a polymeric material comprising organic polymers, inorganic polymers or any combination thereof. In another embodiment, the polymeric material comprises thermoplastic polymers, thermoset polymers or any combination thereof. In another embodiment, the organic polymer comprises hydrogels, polyolefins such as polyvinylchloride (PVC), polyethylene, polystyrene and polypropylene, epoxy resins, acrylate resins such as poly methyl methacrylate, polyurethane or any combination thereof. In another embodiment, the inorganic polymer comprise silicone polymers such as polydimethylsiloxane (PDMS), ceramics, metals or any combination thereof. In another embodiment, the hydrogel is poloxamer or alginate. In another embodiment, the commercial poloxamer is used or it is formed by a reaction between a polymer and other reagent. In another embodiment, the polymer is poly(ethylene glycol) (PEG) with reactive end groups (such as epoxides in PEG-diglycidyl ether) and the reagent has multiple reactive sites (e.g. diethylenetriamine). Each possibility represents a separate embodiment of this invention.

In some embodiments, the weight ratio of the particles to the polymeric material is between 0.25-5%. In another embodiment, the weight ratio is between 0.5-2%. In another embodiment, the weight ratio is between 1-5%.

Another polymer material to be used in the context of this invention is resins used in dental, surgical, chirurgical and orthopedic composite materials. In such applications, anti-microbial particles could be first dispersed within the resin part or added simultaneously with filler or any other solid ingredients (if any). Most of these resins are acrylic or epoxy type monomers that undergo polymerization in-vivo.

In one embodiment, this invention provides a composition comprising the anti-microbial particles of this invention for use in printing. In another embodiment, in 3D printing.

Preparation of the compositions of this invention

In some embodiments, the composites of this invention are prepared by embedding the anti-microbial particles into the polymeric materials of this invention. In another embodiment, one type of particle is embedded in the polymeric materials. In another embodiment, a combination of different particle types is embedded in the polymeric materials. In some embodiments, the embedding may be achieved by a variety of methodologies.

In some embodiments, embedding functionalized microparticles into a polymeric material is obtained by two methodologies: A) Extrusion technology: the particles are added into molten thermoplastic polymer into extruder, preferably twin-coned extruder. B) A thermoplastic or thermoset polymer is heated in an organic solvent (non-limiting examples comprise xylene, toluene, their derivatives or any combination thereof) under reflux conditions to achieve the complete dissolution of the polymer. The anti-microbial particles are then dispersed in the same solvent as used for the polymer and the mixture is added to the dissolved polymer using overhead stirrer or homogenizer. After complete dispersion of particles within the polymer, the solvent is evaporated using conventional distillation or evaporation method.

In some embodiments, embedding functionalized microparticles into a silicone based polymeric material is obtained by several methodologies: A) Room temperature vulcanization (RTV) of silicone precursor is achieved, wherein particles are incorporated into unpolymerized or pre-polymerized silicone before final curing at final concentration of 0.5-8% wt particles/silicone polymer. In another embodiment, the curing is activated by moisture. In another embodiment, the curing is activated by heat. B) RTV of silicone precursor is achieved, wherein polymerization is induced by mixing two components of the polymerization mixture. In another embodiment, particles are incorporated into both parts at final concentration of 0.5-8% wt. particles/silicone polymer, or in one of the parts at doubled concentration, giving the 0.5-8% wt. particles/silicone polymer final concentration.

Thus, according to some embodiments, this invention provides a method for preparing a composition comprising embedding a plurality of anti-microbial particles in a polymeric material as described above, wherein the particles are embedded in the material, the method comprises a step of adding the particles as described above, into a molten polymer material utilizing extrusion or to a polymer solution in solvent or via polymerization with the particles and polymer precursors.

In some embodiments, particles according to this invention are homogeneously distributed on the outer surface of the polymeric material in a surface concentration of between about 0.1 to about 100 particles per sq. micrometer. In another embodiment, particles according to this invention are homogeneously distributed on the outer surface of the polymeric material in a surface concentration of between about 1 to about 100 particles per sq. micrometer. The term “homogeneous distribution” is used to denote a distribution, characterized in that the standard deviation of the number of particles per sq. um is no more than the average number of particles per sq. micrometer. A homogeneous distribution is preferred for reproducibility and product specifications. If the distribution is not even, the product may exhibit different properties at different areas. The distribution of the particles away from the outer surface, that is, their bulk concentration, may be similar to that on the outer surface. As a general rule, the total surface of the particles preferably occupies at most about 20% of the surface of the material, preferably between 1% to 15%, more preferably between 1% and 5% and most about between 1% and 3% of the surface of the material.

According to some embodiments, on the average, every sq. micrometer of the outer surface of polymeric material has at least one particle of this invention.

Compositions and Methods of Use Thereof

According to another aspect of the invention there is provided a method for inhibition of bacteria, by contacting the bacteria with an anti-microbial particle of this invention, or a composition or pharmaceutical composition comprising the particle(s) of this invention. The term “inhibition” is referred to destruction, i.e. annihilation, of at least 99% of the bacteria, preferably 99.9%, most preferably 99.99% of the bacteria; reduction in the growth rate of the bacteria; reduction in the size of the population of the bacteria; prevention of growth of the bacteria; causing irreparable damage to the bacteria; destruction of a biofilm of such bacteria; inducing damage, short term or long term, to a part or a whole existing biofilm; preventing formation of such biofilm; inducing biofilm management; or bringing about any other type of consequence which may affect such population or biofilm and impose thereto an immediate or long term damage (partial or complete).

The term “biofilm” refers to a population of biological species (bacteria) attached to a solid surface.

The terms “anti-microbial” and “anti-bacterial” are used herein interchangeably. The quaternary ammonium and the pyridinium groups of this invention [—⁺N(R₁)(R₂)(R₃), —⁺N(R₁′) (R₂′) (R₃′),

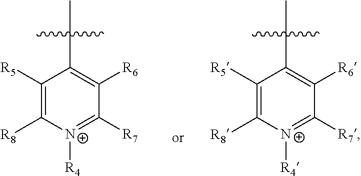

defined in structures (I) and (Ia)] provide the anti-microbial activity. The quaternary ammonium's and pyridinium's activity remains strong at any pH. The ammonium and pyridinium functional groups are not likely to cause undesirable side effects such as irritation of soft tissue, if used in contact with skin or mucosa or if used as a pharmaceutical composition.

In a preferred embodiment, the inhibition is achieved by contacting the bacteria with a matrix containing up to 5% w/w, more preferably up to 1% particles according to this invention, or compositions comprising them.

In one embodiment, this invention further provides a composition or a pharmaceutical composition comprising anti-microbial particles as referred hereinabove. In another embodiment, the composition/pharmaceutical composition comprises one type of particle. In another embodiment, the composition/pharmaceutical composition comprises a combination of different particle types. In one embodiment, non-limiting examples for a composition/pharmaceutical composition of this invention are dental adhesives, bone cement, dental restorative materials such as all types of composite based materials for filling tooth-decay cavities, endodontic filling materials (cements and fillers) for filling the root canal space in root canal treatment, materials used for provisional and final tooth restorations or tooth replacement, including but not restricted to inlays, onlays, crowns, partial dentures (fixed or removable) dental implants, and permanent and temporary cements used in dentistry for various known purposes, dental and orthopedic resin based cements, sealers, composite materials, adhesives and cements, dental restorative composites, bone cements, tooth pastes, lotions, hand-sanitizers, ointments and creams used for dermatology, wound care or in the cosmetic industry, plastic wear for medical and research laboratories; food packaging, mainly for dairy products and fresh meat and fish; pharmaceuticals packaging, paints for ships, that prevent growth of biofilm or treats, breaks down and/or kills a biofilm or bacteria within, paints for bathrooms, paint for hospitals and clean rooms; water filtration media and many others. Each possibility represents a separate embodiment of this invention. In some embodiments, the particles or composition comprising thereof are used for dental and orthopedic resin based cements, sealers, composite materials, adhesinves and cements; for dental and orthopedic metal implants and wires; for surgical sutures; for catheters, metal surgical tools, non-surgical medical devices. Each possibility represents a separate embodiment of this invention.

In one embodiment the composition or composite of this invention is a varnish or glaze which is applied to the tooth surface, a restoration of tooth or a crown comprising the particles of this invention. In another embodiment the varnish or glaze provide a protective coating, lacquer; superficially polished appearance to the tooth surface, restoration or crown of the tooth. In another embodiment, the varnish is a fluoride varnish which is a highly concentrated form of fluoride which is applied to the tooth's surface, as a type of topical fluoride therapy. In another embodiment, the aim of glazing is to seal the open pores in the surface of a fired porcelain. Dental glazes are composed of colorless glass powder, applied to the fired crown surface, so as to produce a glossy surface. Unglazed or trimmed porcelain may also lead to inflammation of the soft tissues it contacts.

In one embodiment, the composition/pharmaceutical composition of this invention is in a form selected from the group consisting of a cream, an ointment, a paste, a dressing and a gel, more preferably, wherein the composition is formulated for topical application or administration. In another embodiment, the composition is intended for administration into an oral cavity. The composition may be formulated as a tooth paste, and/or may be applied to a surface or medical device selected from the group consisting of: a denture cleaner, post hygienic treatment dressing or gel, mucosal adhesive paste, a dental adhesive, a dental restorative composite based material for filling tooth, decay cavities, a dental restorative endodontic filling material for filling root canal space in root canal treatment, a dental restorative material used for provisional and final tooth restorations or tooth replacement, a dental inlay, a dental onlay, a crown, a partial denture, a complete denture, a dental implant and a dental implant abutment.

In one embodiment, the pharmaceutical composition further comprises at least one pharmaceutically active ingredient. In another embodiment, non-limiting examples of pharmaceutically active ingredients include Analgesics, Antibiotics, Anticoagulants, Antidepressants, Anticancers, Antiepileptics, Antipsychotics, Antivirals, Sedatives and Antidiabetics. In another embodiment, non-limiting examples of Analgesics include paracetamol, non-steroidal anti-inflammatory drugs (NSAIDs), morphine and oxycodone. In another embodiment, non-limiting examples of Antibiotics include penicillin, cephalosporin, ciprofolxacin and erythromycin. In another embodiment, non-limiting examples of Anticoagulants include warfarin, dabigatran, apixaban and rivaroxaban. In another embodiment, non-limiting examples of Antidepressants include sertraline, fluoxetine, citalopram and paroxetine. In another embodiment, non-limiting examples of Anticancers include Capecitabine, Mitomycin, Etoposide and Pembrolizumab. In another embodiment, non-limiting examples of Antiepileptics include Acetazolamide, Clobazam, Ethosuximide and lacosamide. In another embodiment, non-limiting examples of Antipsychotics include Risperidone, Ziprasidone, Paliperidone and Lurasidone. In another embodiment, non-limiting examples of Antivirals include amantadine, rimantadine, oseltamivir and zanamivir. In another embodiment, non-limiting examples of Sedatives include Alprazolam, Clorazepate, Diazepam and Estazolam. In another embodiment, non-limiting examples of Antidiabetics include glimepiride, gliclazide, glyburide and glipizide.

In another embodiment, the pharmaceutical composition further comprises excipients. In another embodiment, the excipient comprises binders, coatings, lubricants, flavors, preservatives, sweeteners, vehicles and disintegrants. In another embodiment, non-limiting examples of binders include saccharides, gelatin, polyvinylpyrolidone (PVP) and polyethylene glycol (PEG). In another embodiment, non-limiting examples of coatings include hydroxypropylmethylcellulose, polysaccharides and gelatin. In another embodiment, non-limiting examples of lubricants include talc, stearin, silica and magnesium stearate. In another embodiment, non-limiting examples of disintegrants include crosslinked polyvinylpyrolidone, crosslinked sodium carboxymethyl cellulose (croscarmellose sodium) and modified starch sodium starch glycolate.

In one embodiment, the invention is directed to a packaging composition comprising a thermoplastic polymer and/or hydrogel embedded with anti-microbial particles as referred hereinabove. In another embodiment, the thermoplastic polymer and/or hydrogel is embedded with a mixture of two or more different particles. In another embodiment, the packaging composition is used in the packaging of food, beverage, pharmaceutical ingredients, medical devices, surgical equipment before operation, pre operation equipment, cosmetics, and sterilized equipment/materials.

In one embodiment the packaging composition comprises a thermoplastic polymer and/or hydrogel embedded with the particles as referred hereinabove. In another embodiment, the thermoplastic polymer is polyvinylchloride (PVC), polyethylene, polypropylene, silicone, epoxy resin or acrylic polymers. In another embodiment, the thermoplastic polymer is poly methylmethacrylate or polyurethane.

In another embodiment, the packaging composition further comprises binders, coatings, lubricants and disintegrants. In another embodiment, non-limiting examples of binders include saccharides, gelatin, polyvinylpyrolidone (PVP) and polyethylene glycol (PEG). In another embodiment, non-limiting examples of coatings include hydroxypropylmethylcellulose, polysaccharides and gelatin. In another embodiment, non-limiting examples of lubricants include talc, stearin, silica and magnesium stearate. In another embodiment, non-limiting examples of disintegrants include crosslinked polyvinylpyrolidone, crosslinked sodium carboxymethyl cellulose (croscarmellose sodium) and modified starch sodium starch glycolate.

In one embodiment, the packaging composition is used for packaging pharmaceutical ingredients. In another embodiment, non-limiting examples of pharmaceutical ingredients include analgesics, antibiotics, anticoagulants, antidepressants, anti-cancers, antiepileptics, antipsychotics, antivirals, Sedatives and antidiabetics. In another embodiment, non-limiting examples of analgesics include paracetamol, non-steroidal anti-inflammatory drugs (NSAIDs), morphine and oxycodone. In another embodiment, non-limiting examples of antibiotics include penicillin, cephalosporin, ciprofloxacin and erythromycin. In another embodiment, non-limiting examples of anticoagulants include warfarin, dabigatran, apixaban and rivaroxaban. In another embodiment, non-limiting examples of Antidepressants include sertraline, fluoxetine, citalopram and paroxetine. In another embodiment, non-limiting examples of anti-cancers include Capecitabine, Mitomycin, Etoposide and Pembrolizumab. In another embodiment, non-limiting examples of antiepileptics include Acetazolamide, Clobazam, Ethosuximide and lacosamide. In another embodiment, non-limiting examples of antipsychotics include Risperidone, Ziprasidone, Paliperidone and Lurasidone. In another embodiment, non-limiting examples of antivirals include amantadine, rimantadine, oseltamivir and zanamivir. In another embodiment, non-limiting examples of sedatives include Alprazolam, Clorazepate, Diazepam and Estazolam. In another embodiment, non-limiting examples of antidiabetics include glimepiride, gliclazide, glyburide and glipizide.

In one embodiment, the packaging composition is used in the packaging of food ingredients. In another embodiment, non-limiting examples of food ingredients packaged with the packaging material of the invention include fresh food, preservatives, sweeteners, color additives, flavors and spices, nutrients, emulsifiers, binders and thickeners. In another embodiment, non-limiting examples of fresh food include: meat, poultry, fish, dairy products, fruits and vegetables. In another embodiment, non-limiting examples of preservatives include Ascorbic acid, citric acid, sodium benzoate, calcium propionate, sodium erythorbate, butylated hydroxy toluene (BHT), silver, chlorhexidine, trichlozan and sodium nitrite. In another embodiment, non-limiting examples of sweeteners include Sucrose (sugar), glucose, fructose, sorbitol, mannitol and corn syrup. In another embodiment, non-limiting examples of color additives include Orange B, Citrus Red No. 2, annatto extract, beta-carotene, grape skin extract, cochineal extract or carmine and paprika oleoresin. In another embodiment, non-limiting examples of flavors and spices include monosodium glutamate, glycine slats, inosinic acid, isoamyl acetate, and limonene and allyl hexanoate. In another embodiment, non-limiting examples of nutrients include Thiamine hydrochloride, riboflavin (Vitamin B₂), niacin, niacinamide, folate or folic acid. In another embodiment, non-limiting examples of emulsifiers include Soy lecithin, mono- and diglycerides, egg yolks, polysorbates and sorbitan monostearate. In another embodiment, non-limiting examples of binders and thickeners include Gelatin, pectin, guar gum, carrageenan, xanthan gum and whey.

In one embodiment, this invention provides a method for inhibiting or preventing biofilm formation, comprising applying onto a susceptible or infected surface or a medical device an anti-microbial particle or a composition of this invention.

In another embodiment, this invention provides an anti-microbial particle or a composition of this invention for use in inhibiting or preventing a biofilm formation.

In one embodiment, this invention provides a method for inhibiting or preventing biofilm formation or growth comprising placing a medical device of this invention (comprising a composition and/or anti-microbial particle of this invention as referred hereinabove) on the surface to be treated. In another embodiment, the medical device is a wound dressing. In another embodiment, the wound dressing comprises the anti-microbial particles of this invention and polymers and/or biopolymers. In another embodiment, non-limiting examples of the polymers and/or biopolymers include: carboxy methyl cellulose (CMC), cotton fibres, alginic acid and salts thereof (e.g. Ca/Na), gelatin, collagen, polyesters, nylons and fibres thereof, synthetic hydrogels, poloxamers, polyethylene glycol and polypropylene glycol.

In another embodiment, this invention provides a medical device of this invention for use in inhibiting or preventing biofilm formation or growth.

In one embodiment, this invention provides a method for inhibition of bacteria, the method comprising the step of contacting the bacteria with the pharmaceutical or packaging composition or composite of this invention.

In another embodiment, this invention provides a pharmaceutical or packaging composition or for use in inhibiting bacteria.

In one embodiment, this invention provides a method for treating, breaking down or killing biofilm or bacteria within, comprising applying onto a susceptible or infected surface or a medical device the anti-microbial particle or the pharmaceutical or packaging composition or composite of this invention.

In another embodiment, this invention provides an anti-microbial particle or a composite or a pharmaceutical or packaging composition of this invention for use in treating, breaking down or killing biofilm or bacteria within.

Applications out of the medical field may for example be in clothing (e.g. for sports or outdoor activity; to prevent bacteria-induced sweat odor), athlete shoes or the inner part of a shoe wherein bacteria tend to collect, sportswear and clothing for outdoor activity, tooth brushes and any brush that are in contact with the human body, air and water filters, water treatment and distribution systems, pet cages as well as other veterinary items, etc.

In some embodiments, the anti-microbial compositions or composites of this invention affect annihilation of at least about 99% of the contacted bacteria, preferably, at least about 99.99% of the contacted bacteria.

It was further surprisingly discovered that the particles within compositions/composites/medical devices of this invention maintain high anti-microbial properties over time without leaching out and with no alteration of the properties of the hosting matrix. Such particles demonstrate enhanced anti-bacterial activity originating from the presence of closely packed anti-bacterial groups on a given particle's surface.

Medical Devices of this Invention

In one embodiment, this invention further provides a medical device comprising an anti-microbial particle or a composition of this invention. In one embodiment, non-limiting examples for medical devices of this invention are catheters, stents, surgical mesh, breast implants, joint replacements, artificial bones, artificial blood vessels, artificial heart valves (cardiology), artificial skin, plastic surgery implants or prostheses, intra uterin devices (gynecology), neurosurgical shunts, contact lenses (ophthalmology), intraocular lenses, ocular prosthesis, uretral stents, coating for subcutaneous (such as orthopedic or dental) implants, insulin pumps, contraceptives, pacemakers, tubing and canulas used for intra venous infusion, tubing and canulas used for dialysis, surgical drainage tubing, urinary catheters, endotracheal tubes, wound covering (dressing and adhesive bandage) and treatment (e.g. gels, ointments, pastes and creams for wound care which reduce biofilm and bacteria to aid wound healing) materials, sutures, catheters of all kinds that are inserted temporarily or permanently in blood vessels as well as the urinary system, shunt for use in brain applications, surgical gloves, tips for ear examination, statoscope ends and other elements used by the medical personnel; tooth brushes, tooth pick, dental floss, interdental and tongue brushes, surgical sutures, metal surgical tools, non-surgical medical devices, dental, and orthopedic metal implants and wires and surgical drains, syringes, trays, tips, gloves and other accessories used in common medical and dental procedures. In another embodiment, the wound dressing comprises the anti-microbial particles of this invention and polymers and/or biopolymers. In another embodiment, non-limiting examples of the polymers and/or biopolymers include: carboxy methyl cellulose (CMC), cotton fibres, alginic acid and salts thereof (e.g. Ca/Na), gelatin, collagen, polyesters, nylons and fibres thereof, synthetic hydrogels, poloxamers, polyethylene glycol and polypropylene glycol.

In one embodiment, this invention further provides a medical device comprising a dental appliance. In one embodiment, this invention further provides a medical device comprising an orthodontic appliance. The dental appliance and the orthodontal appliance comprise the particles and composition of this invention. In some embodiments, the orthodontal appliance include an aligner for accelerating the tooth aligning, a bracket, a dental attachment, a bracket auxiliary, a ligature tie, a pin, a bracket slot cap, a wire, a screw, a micro-staple, cements for bracket and attachments and other orthodontic appliances, a denture, a partial denture, a dental implant, a periodontal probe, a periodontal chip, a film, or a space between teeth. In some embodiments, the dental appliance include a mouth guard, used to prevent tooth grinding (bruxer, Bruxism), night guard, an oral device used for treatment/prevention sleep apnea, teeth guard used in sport activities.

In one embodiment, this invention further provides a trans dermal medical device such as orthopedic external fixation screws and wires used for bone fixations and stabilization and trans mucosal elements used in dental implants such as healing caps, abutments (such as multiunit), for screw retained or for cement retained dental prosthesis.

In one embodiment, this invention further provides a medical device comprising an endoscope (rigid and flexible), including, and not limited to a colonoscope, gastroscope, duodenoscope, bronchoscope, cystoscope, ENT scopes, laporoscope, laryngoscope and similar instruments for examination or treatment the inside of the patient's body, including any parts thereof, as well as accessories and other devices used in the procedure which either come in contact with body tissue or fluids; tubes, pumps, containers and connectors (used inside or outside the body) through which fluids, air or gas may be pumped into or suctioned out from the patient and could become contaminated by the patient or transfer contaminants from other patients; items such as brushes, trays, covers, tubes, connectors cabinets and bags used for reprocessing, cleaning, transporting and storing such equipment and can transmit or host biological contaminants, as well as filters for air or water used in dental or medical procedures, hospital surfaces (such as floors, tabletops), drapes, curtains, linen, handles and the like.

The antimicrobial property may protect the patient and the medical staff from cross contamination from patient to patient or from patient to the examiner. Self-sterilizing packaging for medicines and items that enter the operation room are also beneficial.

In one embodiment, this invention further provides processes for preparing the medical devices comprising the composites. In another embodiment, the medical devices are prepared via the steps of: providing a fluid phase of the composite of this invention; shaping the fluid; and hardening of the shaped fluid, affording the desired medical device. In another embodiment, the medical devices are prepared via the steps of: providing a solid phase of the composite; and shaping of the solid, affording the desired medical device. In another embodiment, the shaping is accomplished via extrusion or molding. In another embodiment, fluid phase of the composite comprises melted composite or a composite dissolved in a solvent.

Another polymer material to be used in the context of this invention is resins used in dental, surgical, chirurgical and orthopedic composite materials. In such applications, anti-microbial particles could be first dispersed within the resin part or added simultaneously with filler or any other solid ingredients (if any). Most of these resins are acrylic or epoxy type monomers that undergo polymerization in-vivo.

The following examples are presented in order to more fully illustrate the preferred embodiments of this invention. They should in no way, however, be construed as limiting the broad scope of this invention.

### EXAMPLES

### Example 1

### Thermal Stability Study of Various Anti-Microbial Particles

The thermal stability of particles of this invention was demonstrated in comparative study using differential scanning calorimetry, using ISO 11357. Three different samples were tested: (2QA POSS)—linker is aliphatic chain, hydrophobic group is octyl; (2QA BP)—linker is aliphatic chain, hydrophobic group is benzyl; and (QA BP)—linker is aliphatic chain with hydroxy onto β-carbon and the hydrophobic group is benzyl.
a. (2QA POSS)—linker is aliphatic chain, hydrophobic group is octyl;

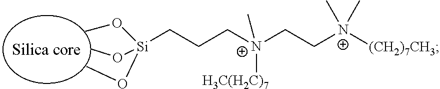

b. (2QA BP)—linker is aliphatic chain, hydrophobic group is benzyl:

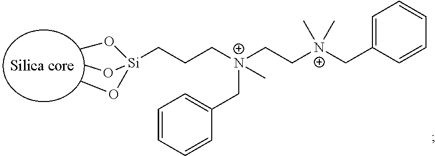 [SMILES: CO[Si](CCC[N+](C)(CC[N+](C)(C)Cc1ccccc1)Cc1ccccc1)(OC)OC]

and
c. (QA BP)—linker is aliphatic chain with hydroxy onto β-carbon and the hydrophobic group is benzyl:

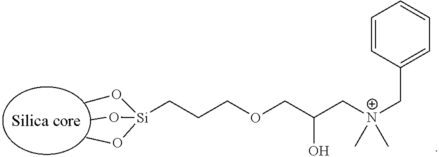 [SMILES: CO[Si](CCCOCC(O)C[N+](C)(C)Cc1ccccc1)(OC)OC]

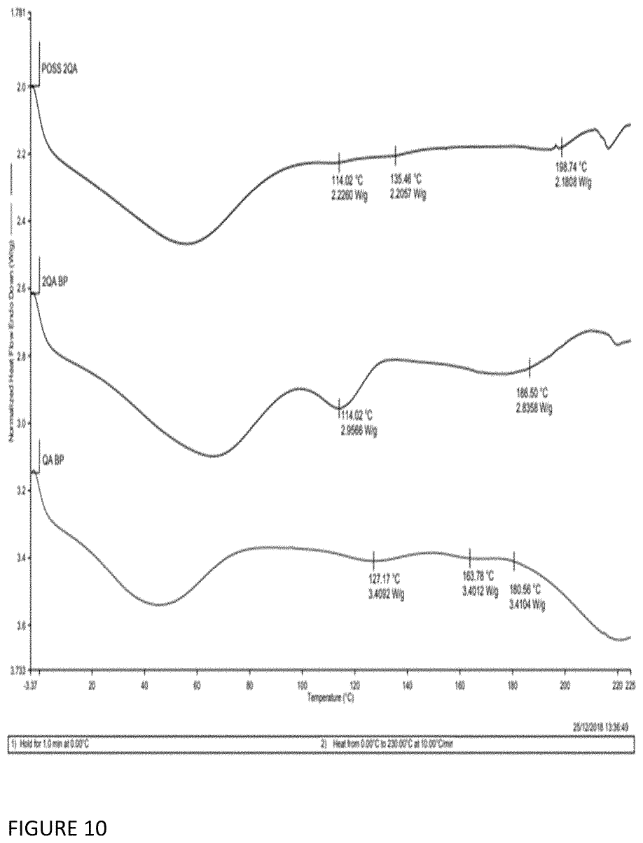

Thermal decomposition of antibacterial particles occurred (FIG. 10) at 114.02° C. for both 2QA POSS and 2QA BP, when only at 127.17° C. for QA BP.

### Example 2

### Anti-Bacterial Activity of Particles Having No Hydrogens in Betta Position to the Quaternary Ammonium

Antibacterial polymeric particles without hydrogens in beta position to quaternary ammonium were prepared by reacting chlorinated crosslinked polystyrene with 2,2-Dimethyl-1,3-propanediamine, then reacted with benzyl chloride and quaternized with methyl iodide to provide the particles below:

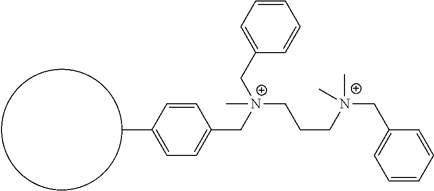 [SMILES: Cc1ccc(C[N+](C)(CCC[N+](C)(C)Cc2ccccc2)Cc2ccccc2)cc1]

Prepared particles were compounded with polypropylene beads at 5 and 10% (w/w), then extruded using twin cone compounder at 220° during 5 min. The obtained extrudate was plastic roads cut to the length of −2 cm.

Antibacterial assay: both compounded samples with 5 and 10% (w/w) of particles and control sample of polypropylene rods without antibacterial particles (control group) were sterilized by immersing into 70% (w/w) solution of ethanol and then irradiated under ultraviolet light for 30 min. Subsequently, all samples were inoculated with 20 μl of Enterococcus faecalis (E. faecalis) suspension in brain heart infusion (BHT) and allowed to dry to ensure physical contact between the sample surface and the bacteria.

Each sample was then rolled onto agar BHT plate and all plates were incubated overnight to allow development of the bacteria colonies.

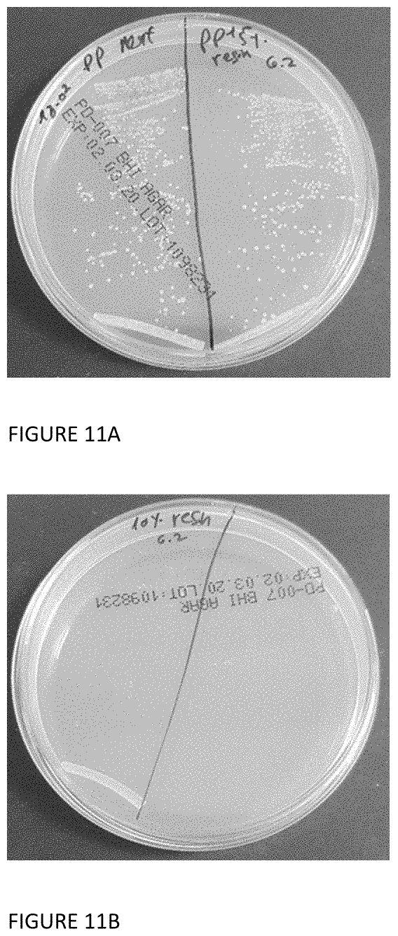

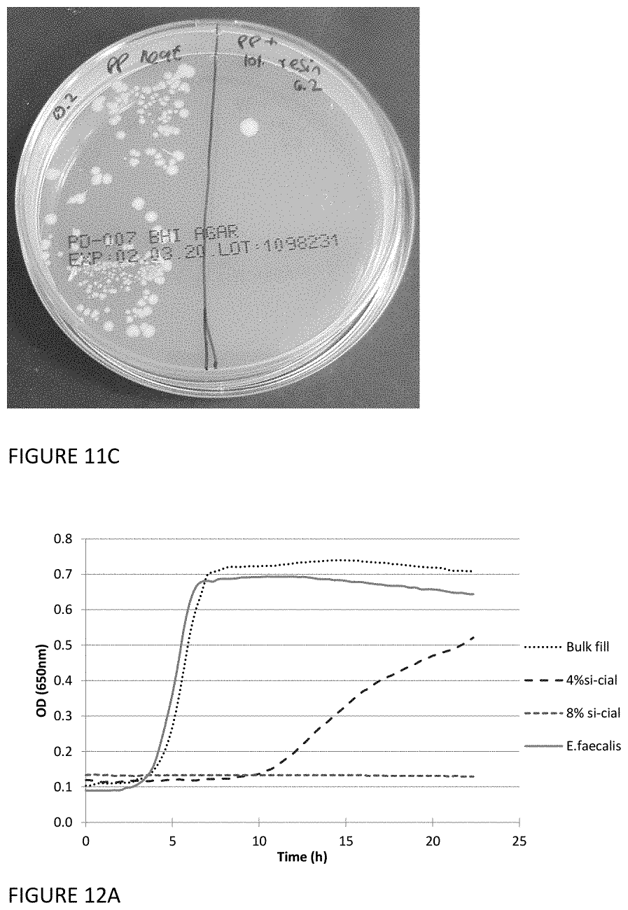

The results are represented in FIGS. 11A-11C and show that increasing anti-bacterial particles concentration leads to higher inhibition of bacterial activity.

### Example 3

### Anti-Bacterial Activity of Particles Having Low Concentration of Hydrogens in Betta Position to the Quaternary Ammonium

Polysilsesquioxane (POSS) antibacterial particles were prepared by reacting 3-aminopropyltrimethoxysilane with 1 eq. of cinnamaldehyde in presence of NaBH₄. The reaction was conducted in dry toluene under continuous water removal using dean-stark device. Subsequently, all the toluene was removed under heat and vacuum. Dry tetrahydrofuran was added as solvent, then 3 eq. of Methyliodide were added to obtain quaternary ammonium. POSS particles were obtained by adding 10% (w/w) NaOH solution in water while stirring for 30 min, followed by POSS precipitation. Obtained POSS particles were freeze-dried then grinded to fine powder. POSS particles were marked as Si-cial and blended with polyvinylchloride (PVC) powder at 4 and 8% (w/w), then extruded at 160° C. for 3 min.

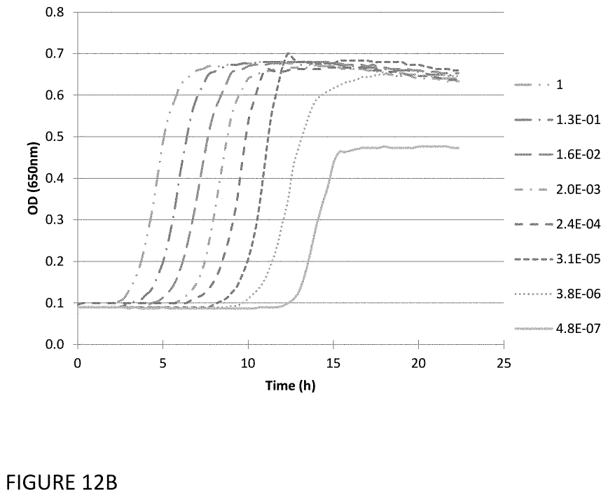

Obtained samples were placed at the sidewall of 96-wells sterile plate and examined under the direct contact test (DCT: Assessment of antibacterial activity of endodontic sealers by a direct contact test. Weiss E., Shalhav M., Fuss Z. Endod. Dent. Traumatol. 1996 August; 12(4):179-84) to evaluate antibacterial activity. Calibration for the DCT was done as represented in FIG. 12B. As FIG. 12A shows, increasing anti-bacterial particles concentration gives rise to higher inhibition of bacterial activity. Already at 4% of particles, bacterial activity was reduced by ˜6 logs where 8% of particles led to complete bacteria inhibition.

While certain features of this invention have been illustrated and described herein, many modifications, substitutions, changes, and equivalents will now occur to those of ordinary skill in the art. It is, therefore, to be understood that the appended claims are intended to cover all such modifications and changes as fall within the true spirit of this invention.

What is claimed is:

1. An anti-microbial particle represented by structure (I): 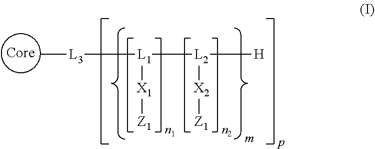
wherein
the core is an organic polymer or an inorganic material;
L₁ is a first linker or a bond;
L₂ is a second linker;
L₃ is a third linker or a bond;
Z₁ is 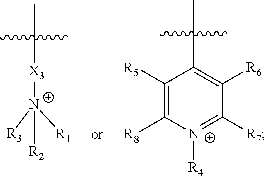
Z₂ is 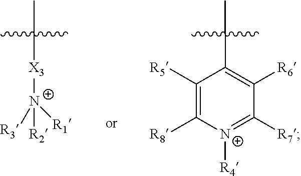
R₁ and R₁′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₂ and R₂′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₃ and R₃′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R₄ and R₄′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ and R₅′ are each independently H, alkyl, terpenoid moiety, cycloalkyl, aryl, heterocycle, a conjugated alkyl, alkenyl, alkynyl or any combination thereof;
R₆ and R₆′ are each independently H, alkyl, terpenoid moiety, cycloalkyl, aryl, heterocycle, a conjugated alkyl, alkenyl, alkynyl or any combination thereof;
R₇ and R₇′ are each independently H, alkyl, terpenoid moiety, cycloalkyl, aryl, heterocycle, a conjugated alkyl, alkenyl, alkynyl or any combination thereof;
R₈ and R₈′ are each independently H, alkyl, terpenoid moiety, cycloalkyl, aryl, heterocycle, a conjugated alkyl, alkenyl, alkynyl or any combination thereof;
X₁ and X₂ are each independently a bond, alkylene, arylene, alkenylene, alkynylene or any combination thereof;
X₃ and X₄ are each independently a bond, —O—C(═O)—, methylene, —O—C(═O)—CH₂—, 2,2-disubstituted C₂-C₂₀ alkylene, arylene, 1-alkenylene, 1-alkynylene, 2-alkenylene, 2-alkynylene or any combination thereof;
R is alkyl, aryl, cycloalkyl, heterocycle or any combination thereof;
p defines the number of anti-microbial active unit per one sq nm (nm²) of the core surface, wherein said density is of between 0.01-30 anti-microbial units per one sq nm (nm²) of the core surface of the particle;
n₁ is each independently an integer between 0 to 200;
n₂ is each independently an integer between 0 to 200;
wherein n₁+n₂≥1; and
m is an integer between 1 to 200 and the repeating unit is the same or different.

2. The anti-microbial particle of claim 1, represented by structure (Ia):

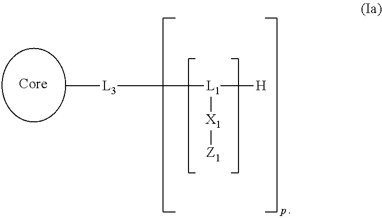

3. The anti-microbial particle of claim 1, represented by structure (II): 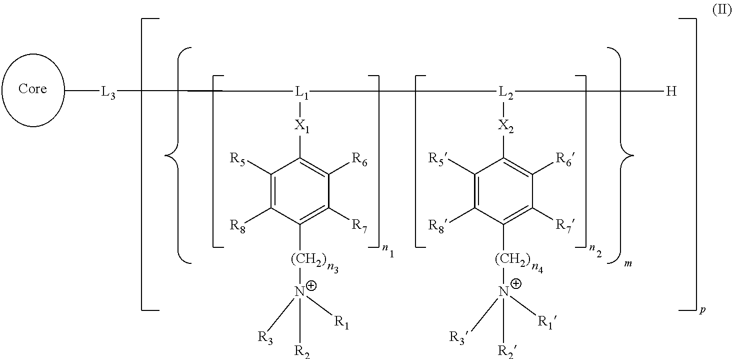
wherein
n₃ and n₄ are each independently 0 or 1.

4. The anti-microbial particle of claim 3, represented by structure (III):

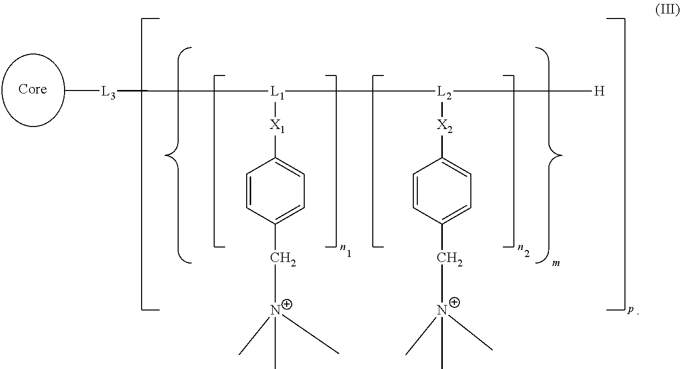

5. The anti-microbial particle of claim 3, represented by structure (IV):

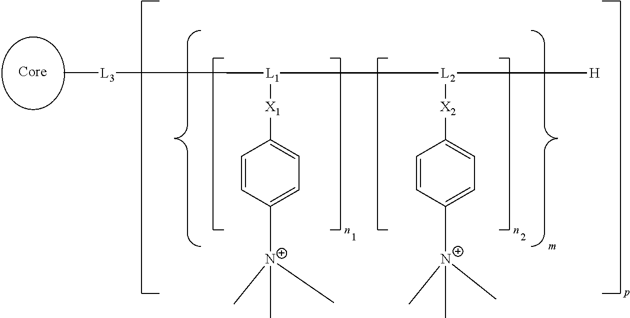

6. The anti-microbial particle of claim 3, represented by structure (V):

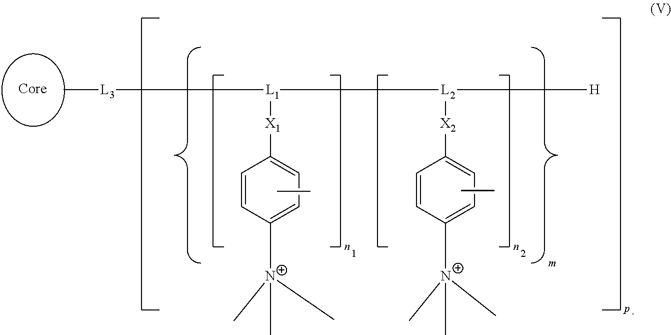

7. The anti-microbial particle of claim 1, represented by structure (VI):

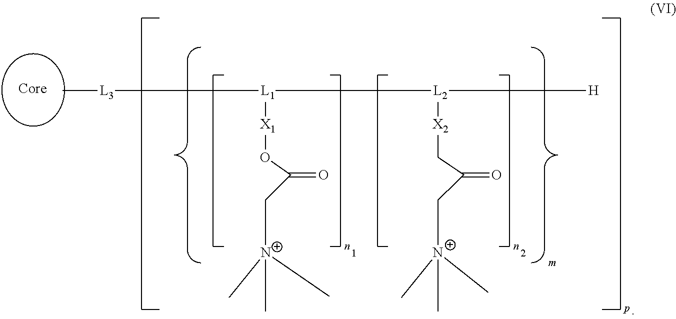

8. The anti-microbial particle of claim 1, represented by structure (VII):

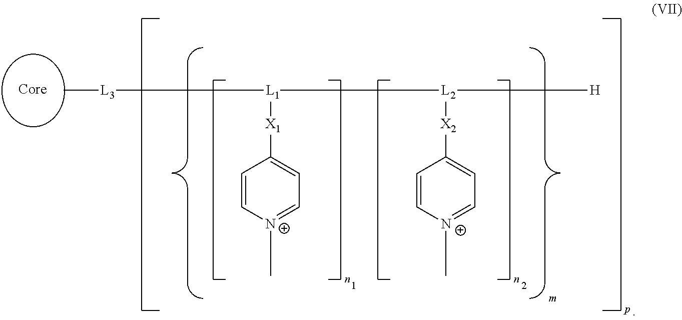

9. A composition comprising a polymeric material and an anti-microbial particle according to claim 1.

10. A method for inhibiting or preventing biofilm formation or growth comprising administering an anti-microbial particle or a composition according to claim 1.

11. A medical device comprising an anti-microbial particle or a composition according to claim 1.

12. The medical device of claim 11, wherein the medical device comprises a composition comprising a polymer material and the anti-microbial particles.

13. The medical device of claim 11, wherein
the medical device is a stent, catheter, surgical drain, surgical mesh or breast implant and the polymer material is silicone based polymer.

14. The medical device of claim 13, wherein the core of the particles comprises silica.

15. The medical device of claim 14, wherein the silica is polyhedral oligomeric silsesquioxane (POSS), amorphous silica, dense silica, aerogel silica, porous silica, mesoporous silica and fumed silica.

16. The medical device of claim 12, wherein the medical device is a stent, catheter, surgical drain, surgical mesh or breast implant and the polymer material is silicone based polymer.

17. The medical device of claim 16, wherein the core of the particles comprises silica.

18. The medical device of claim 17, wherein the silica is polyhedral oligomeric silsesquioxane (POSS), amorphous silica, dense silica, aerogel silica, porous silica, mesoporous silica and fumed silica.

19. A composition comprising a polymeric material and an anti-microbial particle according to claim 2.

20. A composition comprising a polymeric material and an anti-microbial particle according to claim 3.

21. A method for inhibiting or preventing biofilm formation or growth comprising administering an anti-microbial particle or a composition according to claim 2.

22. A method for inhibiting or preventing biofilm formation or growth comprising administering an anti-microbial particle or a composition according to claim 3.
